(19) United States
(12) Patent Application Publication
(10) Pub. No.: US 2004/0242665 A1
(43) Pub. Date: Dec. 2, 2004

(54) Hair composition containing a styrylpyrazole compound, and use thereof for stimulating or inducing hair or eyelash growth and/or reducing loss thereof

(75) Inventors: Christophe Boulle, Lagny S/Marne (FR); Roger Rozot, Lagny S/Marne (FR)

Correspondence Address: BURNS DOANE SWECKER & MATHIS L L P, POST OFFICE BOX 1404, ALEXANDRIA, VA, 22313-1404, US

(73) Assignee: SOCIETE L'OREAL S.A., PARIS (FR)

(21) Appl. No.: 10/694,892

(22) Filed: Oct. 29, 2003

Related U.S. Application Data
(60) Provisional application No. 60/425,276, filed on Nov. 12, 2002.

(30) Foreign Application Priority Data
Oct. 29, 2002 (FR) 02/13522

(51) Int. Cl. A61K 31/415

(52) U.S. Cl. 514/406

(57) ABSTRACT

Haircare compositions containing, formulated into physiologically acceptable media therefor, effective amounts of a styrylpyrazole compound of formula (I), or salt thereof, to stimulate or induce hair growth and/or to reduce loss thereof:

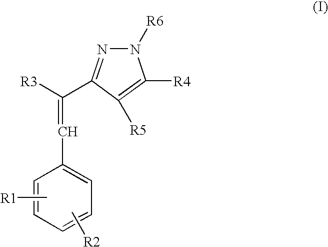

### FIELD OF THE INVENTION

The invention relates to a haircare composition containing an effective amount of a pyrazole compound and more especially of a styrylpyrazole, which is intended to induce and/or stimulate the growth of keratin fibres, especially human keratin fibres, and/or to reduce their loss. The invention also relates to a cosmetic treatment process and to novel styrylpyrazole compounds for stimulating the growth of keratin fibres and/or reducing their loss.

The human keratin fibres to which the invention applies are especially head hair, the eyebrows, the eyelashes, beard hair, moustache hair and pubic hair. The invention applies more especially to human head hair and/or eyelashes.

In particular, the invention relates to a makeup or care composition for the hair or the eyelashes, containing an effective amount of a styrylpyrazole compound, which is intended to increase their density and/or improve their appearance.

### BACKGROUND OF THE INVENTION

Hair growth and hair renewal are mainly determined by the activity of the hair follicles and of their matrix environment. Their activity is cyclical and comprises essentially three phases, namely the anagenic phase, the catagenic phase and the telogenic phase.

The anagenic phase (active phase or growth phase), which lasts several years and during which the hair gets longer, is followed by a very short and transient catagenic phase which lasts a few weeks. During this phase, the hair undergoes a change, the follicle becomes atrophied and its dermal implantation appears higher and higher.

The terminal phase or telogenic phase, which lasts a few months, corresponds to a resting phase of the follicle and the hair ends up by falling out. At the end of this rest period, a new follicle is regenerated in situ and another cycle begins.

The head of hair is thus under permanent renewal, and, out of the approximately 150,000 hairs that make up a head of hair, about 10% are at rest and will be replaced within a few months.

The natural loss or falling-out of the hair may be estimated, on average, as being a few hundred hairs per day for a normal physiological state. This process of permanent physical renewal undergoes a natural change during ageing: the hairs become finer and their cycles shorter.

In addition, various causes may result in a substantial, temporary or permanent loss of hair. This may be loss and impairment of hair at the terminal stage of pregnancy (post-partum), during states of dietary malnutrition or imbalance, or during states of asthenia or of hormonal dysfunction, as may be the case during or at the terminal stage of the menopause. It may also be a case of loss or impairment in the hair related to seasonal phenomena.

It may also be a matter of alopecia, which is essentially due to a disturbance in hair renewal, resulting, in a first stage, in acceleration of the frequency of the cycles to the detriment of the quality of the hair, and then of their quantity. The successive growth cycles result in hairs that are finer and finer and shorter and shorter, gradually transforming into an unpigmented down, thus resulting in a progressive impoverishment of the head of hair. Certain areas are preferentially affected, especially the temporal or frontal lobes in men, and a diffuse alopecia of the crown of the head in women.

The term alopecia also covers a whole family of afflictions of hair follicles whose final consequence is the permanent, partial or general loss of the hair. This is more particularly a matter of androgenic alopecia. In a large number of cases, early loss of hair occurs in genetically predisposed individuals; this is then a matter of androchronogenetic alopecia. This form of alopecia especially affects men.

It is moreover known that certain factors, such as hormonal imbalance, physiological stress or malnutrition, can accentuate the phenomenon.

In certain dermatoses of the scalp with an inflammatory component, for instance psoriasis or seborrhoeic dermatitis, hair loss may be greatly accentuated or may result in highly disrupted follicular cycles.

The cosmetics and pharmaceutical industries have for many years been investigating compositions for eliminating or reducing alopecia, and especially for inducing or stimulating hair growth or reducing its loss.

In this perspective, a large number of compositions comprising very diverse active agents have already been proposed, for instance 2,4-diamino-6-piperidinopyrimidine 3-oxide, or “minoxidil” described in patents U.S. Pat. No. 4,139,619 and U.S. Pat. No. 4,596,812, or the numerous derivatives thereof such as those described, for example, in documents EP 0 353 123, EP 0 356 271, EP 0 408 442, EP 0 522 964, EP 0 420 707, EP 0 459 890 and EP 0 519 819.

Clinical studies have shown that PGF2-α analogues have the property of inducing the growth of body hairs and eyelashes in man and animals (Murray A. and Johnstone M. D., 1997, Am. J. Opht., 124(4), 544-547). In man, tests performed on the scalp have shown that a prostaglandin E2 analogue (viprostol) has the property of increasing the hair density (Roenigk H H., 1988, Clinic Dermatol., 6(4), 119-121).

Moreover, documents WO 98/33497 describes pharmaceutical compositions containing prostaglandins or prostaglandin derivatives, for combating hair loss in man. Prostaglandins of the type A2, F2α and E2 are mentioned as being preferred.

However, prostaglandins are molecules with a very short biological half-life, which act in an autocrine or paracrine manner, this reflecting the local and labile nature of the metabolism of prostaglandins (Narumiya S. et al., 1999, Physiol. Rev., 79(4), 1193-1226).

It is thus seen to be important, in order to maintain and/or increase the hair density in man, to preserve the endogenous reserves of PGF2-α and similarly of PGE2 in various compartments of the hair follicle or its immediate cutaneous environment.

One solution that gives good results is the use of lipoxygenase-inhibiting compounds and/or cyclooxygenase-inducing compounds to promote hair growth; one theory is that the use of such compounds directs the metabolism of fatty acids towards the endogenous synthesis of prostaglandins in preference to other routes.

However, to further improve the results, it would be desirable to be able to prolong the activity of the prostaglandins involved in growing the hair and keeping it alive.

It is moreover well known that the programmes of differentiation of the keratinocytes of the epidermis and of the hair follicle are clearly different. Thus, it is known that the keratins of the hair stalk represent a family (Langbein et al., 2001, J. Biol. Chem. 276: 35123-35132) that is different from the one expressed in the epidermis, that differentiation markers such as the keratins K₁ and K₁₀ are not expressed in the hair follicle and in particular in the outer sheath (Lenoir et al., 1988, Dev. Biol. 130: 610-620), that trichohyalin (O'Guin et al., 1992, J. Invest. Dermatol. 98: 24-32) and keratin K6irs (Porter et al., 2001, Br. J. Dermatol. 145: 558-568) are expressed in the hair follicle, in particular in the inner sheath, but not in the epidermis, and that type-1 cyclooxygenase, although expressed in the epidermis, is not expressed in the keratinocytes of the hair follicle but in the dermal papilla (Michelet, et al., 1997, J. Invest. Dermatol. 108: 205-209).

The Applicant has now demonstrated that an enzyme specifically involved in the degradation of these prostaglandins is present in the dermal papilla of the hair, which is a compartment that is a decisive factor in the life of a hair. Specifically, the Applicant has now proven the presence of 15-hydroxyprostaglandin dehydrogenase (abbreviated as 15-PGDH) at this level. The Applicant has also shown that the inhibition of 15-PGDH has a beneficial effect on hair growth.

Consequently, the present invention relates to a care or treatment composition for human keratin fibres, and especially hair fibres, containing at least one particular inhibitor of 15-hydroxyprostaglandin dehydrogenase and a physiologically acceptable medium.

15-PGDH is a key enzyme in the deactivation of prostaglandins, in particular of PGF2-α and PGE2, which are important mediators of hair growth and survival. It corresponds to the classification EC 1.1.1.141 and is NAD⁺-dependent. It has been isolated from pig kidney; its inhibition with a thyroid hormone, triiodothyronine, at doses very much higher than the physiological doses, has especially been observed.

However, it has never been proposed to use a 15-PGDH inhibitor to maintain and/or increase the density of human keratin fibres and especially the hair density and/or to reduce the heterogeneity of the diameters of the keratin fibres and especially of the hair in man. The expression “increase the density of keratin fibres, and especially the hair density” means increasing the number of keratin fibres, and especially of hairs per cm² of skin or of scalp.

### SUMMARY OF THE INVENTION

The Applicant has found that certain pyrazole compounds, and especially certain salified or non-salified styrylpyrazoles, are, surprisingly, endowed with favourable activity towards improving the density of human keratin fibres and especially the hair density. The applicant has moreover found that these compounds are inhibitors of 15-hydroxyprostaglandin dehydrogenase.

One subject of the present invention is thus a care and/or makeup composition for keratin fibres, especially human keratin fibres, and more especially a haircare or mascara composition for topical application containing, in a physiologically acceptable medium, an effective amount of a styrylpyrazole compound of formula (I), or a salt thereof:

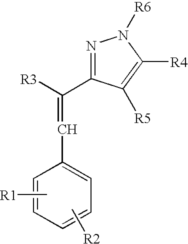

in which:

R₁ R₂, R₄ and R₅, which may be identical or different, are chosen from hydrogen, a halogen, groups OR₇, SR₇, NR₇R′₇, COOR₇, CONR₇R′₇, CF₃, CN, NR₇COR′₇, SO₂R₇, SO₂NR₇R′₇, NR₇SO₂R′₇, COR₇, CSR₇, OCOR₇, COSR₇, SCOR₇, CSNR₇R′₇, NR₇CONR′₇R″₇, NR₇C(═NR′₇)NR″₇R″′₇, NR₇CSR′₇ and NR₇CSNR′₇R″₇, saturated or unsaturated, linear or branched C₁-C₂₀ alkyl radicals, saturated or unsaturated rings of 4 to 7 atoms, optionally containing at least one hetero atom, these rings possibly being separate or fused, the alkyl radicals and the rings also possibly being substituted with at least one substituent A₁, with R₇, R′₇, R″₇ and R″′₇ independently denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₂;

R₃ is chosen from CN, COOR₈, CONR₈R′₈, COR₈, SO₂R₈ and SO₂NR₈R′₈, with R₈ and R′₈ independently denoting hydrogen, a linear or branched C₁-C₂₀ alkyl radical or a ring of 4 to 7 atoms, isolated or fused to another ring and optionally containing at least one hetero atom, the alkyl radical or the said rings being saturated or unsaturated and optionally substituted with at least one substituent A₃;

R₆ is chosen from hydrogen, groups COOR₉, COR₉, CSR₉, COSR₉, CONR₉R′₉, SO₂R₉ and SO₂NR₉R′₉, linear or branched, saturated or unsaturated C₁-C₂₀ alkyl radicals and saturated or unsaturated rings of 4 to 7 atoms, optionally containing at least one hetero atom, these rings possibly being separate or fused, the alkyl radicals and the rings also possibly being substituted with at least one substituent A₄, with R₉ and R′₉, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₅;

A₁, A₂, A₃, A₄ and A₅ being chosen independently from halogens, groups OR₁₀, SR₁₀, NR₁₀R′₁₀, COOR₁₀, CH₂COOR₁₀, CONR₁₀R′₁₀, CF₃, CN, NR₁₀COR′₁₀, SO₂R₁₀, SO₂NR₁₀R′₁₀, NR₁₀SO₂R′₁₀, COR₁₀, CSR₁₀, OCOR₁₀, COSR₁₀, SCOR₁₀, CSNR₁₀R′₁₀, NR₁₀CONR′₁₀R′₁₀, NR₁₀C(═NR¹⁰)NR″₁₀R″₁₀, NR₁₀CSNR′₁₀R″₁₀ and NR₁₀CSR′₁₀, with R₁₀, R′₁₀, R″₁₀ and R″′₁₀, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated.

The invention also relates to the use, especially the cosmetic use, of at least one pyrazole compound of formula (I) or a salt thereof, as defined above, as an agent for inducing and/or stimulating the growth of keratin fibres, especially human keratin fibres such as human eyelashes and hair, and/or for reducing their loss and/or for increasing their density.

The invention also applies to the keratin fibres of mammalian animals (for example dogs, horses or cats).

The invention also relates to the cosmetic use of at least one pyrazole compound of formula (I), or a salt thereof, in a cosmetic care and/or makeup composition for human keratin fibres to induce and/or stimulate their growth, to reduce their loss and/or to increase their density and/or to treat androgenic alopecia, and also to the use of at least one compound of formula (I), or a salt thereof, for the preparation of a care or treatment composition for human keratin fibres, which is intended to induce and/or stimulate the growth of the fibres and/or to reduce their loss and/or to increase their density and/or to treat androgenic alopecia.

The human keratin fibres to which the invention applies are especially head hair, the eyebrows, the eyelashes, beard hair, moustache hair and pubic hair. The invention applies more especially to human head hair and/or eyelashes.

The invention also relates to the cosmetic use of at least one pyrazole compound of formula (I), or a salt thereof, in a human cosmetic haircare composition for reducing hair loss and/or for increasing its density. A subject of the invention is also the use of at least one pyrazole compound of formula (I), or a salt thereof, for the preparation of a human hair composition, which is intended to induce and/or stimulate hair growth and/or reduce its loss and/or increase its density.

In particular, the invention relates to the cosmetic use of at least one pyrazole compound of formula (I), or a salt thereof, in a human cosmetic haircare composition or for the preparation of a human hair composition for treating or which is intended to treat alopecia of natural origin and in particular androgenic or andro-chrono-genetic alopecia. Thus, this composition makes it possible to keep the head of hair in good condition and/or to combat natural hair loss and more especially that of humans.

A subject of the invention is also the cosmetic use of at least one pyrazole compound of formula (I), or a salt thereof, in a cosmetic care and/or makeup composition for human eyelashes, to induce and/or stimulate the growth of the eyelashes and/or to increase their density, and also the use of at least one compound of formula (I), or a salt thereof, for the preparation of a care and/or treatment composition for human eyelashes, which is intended to induce and/or stimulate the growth of the eyelashes and/or to increase their density. This composition thus makes it possible to keep the eyelashes in good condition and/or to improve their condition and/or appearance.

A subject of the invention is also a cosmetic process for treating keratin fibres (especially hair or eyelashes) and/or the skin from which the said fibres emerge, including the scalp and the eyelids, which is intended in particular to stimulate the growth of human keratin fibres and/or to reduce their loss, characterized in that it consists in applying to the keratin fibres and/or skin from which the said fibres emerge a cosmetic composition comprising an effective amount of at least one compound of formula (I), or a salt thereof, leaving this composition in contact with the keratin fibres and/or the skin from which the said fibres emerge, and optionally rinsing the fibres and/or the said skin.

This treatment process has the characteristics of a cosmetic process in that it makes it possible to improve the aesthetics of keratin fibres by giving them greater vigour and an improved appearance. In addition, it may be used daily for several months, without medical prescription.

Thus, a subject of the present invention is also a cosmetic process for treating the hair and/or the scalp, which is intended to stimulate the growth of human hair and/or to reduce its loss, characterized in that it consists in applying to the hair and/or the scalp a cosmetic composition comprising an effective amount of at least one compound of formula (I), or a salt thereof, leaving the composition in contact with the hair and/or the scalp, and optionally rinsing the hair and/or the scalp.

This treatment process has the characteristics of a cosmetic process in that it makes it possible to improve the aesthetics of the hair by giving it greater vigour and an improved appearance. In addition, it may be used daily for several months.

More especially, a subject of the present invention is a cosmetic care process for human hair and/or the scalp, to improve their condition and/or appearance, characterized in that it consists in applying to the hair and/or the scalp a cosmetic composition comprising an effective amount of at least one compound of formula (I), or a salt thereof, leaving the composition in contact with the hair and/or scalp and optionally rinsing the hair and/or the scalp.

A subject of the invention is also a cosmetic care and/or makeup process for human eyelashes, to improve their condition and/or appearance, characterized in that it consists in applying to the eyelashes and/or the eyelids a mascara composition comprising at least one compound of formula (I), or a salt thereof, and leaving the composition in contact with the eyelashes and/or the eyelids. This mascara composition may be applied alone or as a basecoat for a standard pigmented mascara, and may be removed like a standard pigmented mascara.

A subject of the invention is also a care or makeup composition for keratin fibres, comprising, in a physiologically acceptable medium, in particular a cosmetic medium, at least one compound of formula (I), or a salt thereof, and at least one additional active agent for promoting the regrowth of human keratin fibres and/or for limiting their loss, chosen from aminexil, FP receptor agonists and vasodilators, and more especially chosen from aminexil, minoxidil, latanoprost, butaprost and travoprost.

A subject of the invention is also the cosmetic use of at least one styrylpyrazole compound of formula (I), or a salt thereof, in a cosmetic composition, as an agent for preserving the amount and/or activity of the prostaglandins in the hair follicle.

A subject of the invention is also the use of at least one styrylpyrazole compound of formula (I), or a salt thereof, for the manufacture of a composition for preserving the amount and/or activity of prostaglandins in the hair follicle.

A subject of the invention is also the use of at least one pyrazole compound of formula (I), or a salt thereof, as an inhibitor of the 15-hydroxyprostaglandin dehydrogenase of human skin. A subject of the invention is also the use of at least one pyrazole compound of formula (I), or a salt thereof, for the manufacture of a composition for treating disorders associated with 15-hydroxyprostaglandin dehydrogenase, especially in humans.

### DETAILED DESCRIPTION OF THE EMBODIMENTS OF THE INVENTION

The term “15-hydroxyprostaglandin dehydrogenase inhibitor” means a compound of formula (I) that is capable of inhibiting or reducing the activity of the enzyme 15-PGDH, especially in man, and/or capable of inhibiting, reducing or slowing down the reaction catalysed by this enzyme.

According to one advantageous embodiment of the invention, the compound of formula (I) is a specific inhibitor of 15-PGDH; the term “specific inhibitor” means a compound of formula (I) that has little or no inhibitory effect on the synthesis of prostaglandins, in particular on the synthesis of PGF2-α or PGE2. According to one particular embodiment of the invention, the 15-PGDH inhibitor has little or no inhibitory effect on the synthesis of prostaglandins, in particular on the synthesis of PGF2-α or PGE2. According to one particular embodiment of the invention, the 15-PGDH inhibitor has little or no inhibitory effect on prostaglandin synthase (PGF synthase).

Specifically, the Applicant has now found that PGF synthase is also expressed in the dermal papilla. Maintaining an effective amount of prostaglandins at the site of action thus results from a complex biological equilibrium between the synthesis and degradation of these molecules. The exogenous supply of compounds that inhibit catabolism will therefore be less effective if this activity is combined with an inhibition of the synthesis.

In the text hereinbelow, and unless specifically mentioned, the use of the term “compound of formula (I)” should be understood as meaning the compound of formula (I) both in nonionic form and in salt form.

Advantageously, the compounds of formula (I) show inhibitory activity on 15-PGDH that is higher than the inhibitory activity on PGF synthase. In particular, the ratio between the inhibitory activities on PGF synthase and on 15-PGDH, respectively, for a given concentration, determined especially by the concentrations that inhibit 50% of the enzymatic activity, respectively, of PGF synthase, IC_{50fs}, and of 15-PGDH, IC_{50dh}, is at least greater than 1, especially at least 3:1 and advantageously greater than or equal to 5:1. The preferred compounds of the invention have an IC_{50fs}/IC_{50dh} ratio of greater than or equal to 10:1 and in particular greater than or equal to 15.

According to the invention, the term “at least one” means one or more (2, 3 or more). In particular, the composition may contain one or more compounds of formula (I). This or these compound(s) may be cis or trans or Z or E isomers, or a mixture of cis/trans or Z/E isomers. In particular the aromatic ring may be in the cis or trans or Z or E position and better still in the Z position relative to the pyrazole ring. This or these compound(s) may also be in tautomeric form. They may also be enantiomers and/or diastereoisomers or a mixture of these isomers, in particular a racemic mixture.

According to the invention, the rings used for R₁ to R₁₀, R′₇, R|₇, R═|₇, R′₈, R′₉, R′₁₀, R″₁₀ and R″′₁₀ contain from 4 to 7 atoms and better still 5 or 6 atoms. They may be saturated or unsaturated and may optionally comprise one or more hetero atoms such as S, N or O or combinations thereof. They may be alone or fused to another ring, which may be identical or different. As saturated carbon-based rings that may be used, mention may be made of the cyclopentyl or cyclohexyl radical. Heterocycles that may be mentioned include pyridine, piperidine, morpholine, pyrrol, furan and thiazole rings. Unsaturated carbon-based rings that may be mentioned include the phenyl or naphthyl radical. In addition, these rings may be substituted with a substituent having the definition given above for A₁. Advantageously, when R₆ comprises one or more hetero atoms, the bond with the nitrogen of the pyrazole ring is in the form of an N—C bond.

According to one embodiment of the invention, the substituent(s) borne by the alkyl or aryl radicals, i.e. A₁ to A₅, are halogen atoms and especially chlorine, bromine, iodine or fluorine atoms, preferably chlorine atoms or linear or branched C₁-C₂₀ alkyl radicals, or alternatively perfluoroalkyl radicals. As an example of perfluoroalkyl radicals that may be used, mention may be made of CF₃.

For the purposes of the invention, the term “alkyl radical” means a hydrocarbon-based radical which may be linear or branched, and saturated or unsaturated. The alkyl radical preferably contains from 1 to 10 carbon atoms.

As examples of alkyl radicals that may be used according to the invention, mention may be made of methyl, ethyl, isopropyl, n-butyl, n-hexyl, 2-ethylhexyl, ethylene and propylene.

According to the invention, the compounds of formula (I) (or the salt(s) thereof) are in isolated form, i.e. non-polymeric. In addition, R₁ and R₂ may be located in any position on the phenyl ring and in particular in a position ortho to the branching of the pyrazole portion.

Preferably at least one from among R₁ and R₂ represents a hydrogen atom, OR₇, CF₃, a halogen atom and especially a chlorine atom, R₇ representing a C₁-C₁₀ alkyl radical, for example methyl. In particular, R₁ and/or R₂ represent(s) a halogen atom, especially chlorine.

Advantageously, R₃ represents CN, COOR₈, CONR₈R′₈ or COR₈, for example CN.

According to one embodiment of the invention, R₄, R₅ and R₆ represent, independently of each other, a C₁-C₁₀ alkyl radical optionally substituted with OR₁₀, for instance CH₂CH₂OR₁₀, NH₂, H, CN, or a saturated or unsaturated hydrocarbon-based ring, for instance a phenyl ring, R₁₀ representing, for example, H. Advantageously, R₆ represents CH₂CH₂OR₁₀ and in particular CH₂CH₂OH or a phenyl radical. Preferably, R₄ represents NH₂ or H. According to one advantageous embodiment, R₅ represents CN or H.

According to the invention, the expression “salts of a compound of formula (I)” means the organic or mineral salts of a compound of formula (I).

As mineral salts that may be used according to the invention, mention may be made of the sodium or potassium salts, and also the zinc (Zn²⁺), calcium (Ca²⁺), copper (Cu²⁺), iron (Fe²⁺), strontium (Sr²⁺), magnesium (Mg²⁺), ammonium and manganese (Mn²⁺) salts; hydroxides, carbonates, halides (chlorides), sulphates, nitrates and phosphates.

The organic salts that may be used according to the invention are, for example, the triethanolamine, monoethanolamine, diethanolamine, hexadecylamine, N,N,N′,N′-tetrakis(2-hydroxypropyl)ethylenediamine and tris(hydroxymethyl)aminomethane salts.

According to one particular embodiment of the invention, the pyrazole compounds to which the invention applies are of formula (II), or a salt thereof:

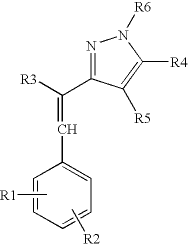

in which:

R₁, R₂, R₄ and R₅ independently represent H, a halogen, OR₇, SR₇, NR₇R′₇, COOR₇, CONR₇R′₇, CF₃, CN, a saturated or unsaturated C₁-C₁₀ alkyl radical, a saturated or unsaturated ring, separate or fused to another ring, optionally containing at least one hetero atom, the alkyl radicals and the rings also possibly being substituted with at least one substituent A₁, with R₇ and R′₇ independently denoting H, a C₁-C₁₀ alkyl radical or a ring which is isolated or fused to another ring;

R₃ represents CN, COOR₈, CONR₈R′₈ or COR₈, with R₈ and R′₈ independently denoting H, a C₁-C₁₀ alkyl radical or a ring which is isolated or fused to another ring and optionally containing at least one hetero atom, the said rings being saturated or unsaturated and optionally substituted with at least one substituent Al;

R₆ represents hydrogen, COOR₉, COR₉, a saturated or unsaturated C₁-C₁₀ alkyl radical or a saturated or unsaturated ring, which is separate or fused to another ring, optionally containing at least one hetero atom, the alkyl radicals and the rings also possibly being substituted with at least one substituent Al, with R₉ and R′₉ independently denoting H, a C₁-C₂₀ alkyl radical or a ring which is isolated or fused to another ring;

the rings containing 5 or 6 atoms;

the hetero atoms being O, N or S or a combination thereof.

Advantageously, the compound of formula (I) or (II) is of Z form.

According to another embodiment of the invention, the pyrazole compounds are of formula (III) below, or a salt thereof:

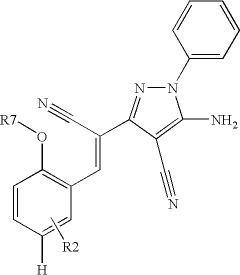

R₇ represents

a) a linear or branched, saturated or unsaturated C₁-C₁₀ alkyl radical, optionally substituted with at least one substituent A₁; or

b) a saturated or unsaturated ring C¹ of 4 to 7 atoms, optionally containing at least one hetero atom and/or being optionally substituted with at least one substituent A₁ and/or optionally fused to at least one saturated or unsaturated ring C² of 4 to 7 atoms, optionally containing at least one hetero atom;

R₂ represents

OR₇, SR₇, NR₇R′₇, COOR₇, CONR₇R′₇, CF₃, CN, NR₇COR′₇, SO₂R₇, SO₂NR₇R′₇, NR₇SO₂R′₇, COR₇, CSR₇, OCOR₇, COSR₇, SCOR₇, CSNR₇R′₇, NR₇CONR′₇R″₇, NR₇C(═NR′₇)NR″₇R″′₇, NR₇CSR′₇ and NR₇CSNR′₇R″₇, a saturated or unsaturated C₁-C₁₀ alkyl radical, a saturated or unsaturated ring C³, which is separate or fused to another ring C⁴, optionally containing at least one hetero atom, the alkyl radicals and the rings also possibly being substituted with at least one substituent A₁ in which R₇ and R′₇, which may be identical or different, denote:

a hydrogen atom or a linear or branched, saturated or unsaturated C₁-C₁₀ alkyl radical,

a C₅ aromatic ring optionally including at least one hetero atom, optionally substituted with at least one substituent A₂; and

in which the hetero atoms are chosen from N, O and S and a combination thereof.

Since the compounds of formula (III) or the salt thereof are novel, a subject of the invention is also a styrylpyrazole compound of formula (III), or a salt thereof.

Advantageously, R₂ represents OR₇ and R₇ represents a saturated C₁-C₁₀ alkyl radical such as methyl.

The compounds of formula (I) or the salts thereof, some of which are known per se, may be manufactured by condensation of a benzaldehyde, optionally substituted with a pyrazole substituted with an activated methylene, with a function from among nitrile, acid, ester, amide and ketone. The removal of water is performed simultaneously by azeotropic distillation and installation of Dean-Stark apparatus. This type of preparation is known to those skilled in the art from document EP 0 245 825. These compounds are in solid form and in particular in pulverulent form, or alternatively in liquid form.

To the Applicant's knowledge, no prior art document describes or suggests that the pyrazole compounds of formula (I) or a salt thereof have the property of inducing and/or stimulating the growth of human keratin fibres, and in particular the hair and the eyelashes, and/or of reducing their loss, or that these compounds can be used topically to increase the density of the keratin fibres (especially the hair and eyelashes).

The effective amount of a compound of formula (I) or a salt thereof corresponds to the amount required to obtain the desired result (i.e. to increase the density of keratin fibres such as the hair and the eyelashes). A person skilled in the art is thus capable of evaluating this effective amount, which depends on the nature of the compound used, the person on whom it is applied and the time of this application.

In the text hereinbelow, and unless otherwise mentioned, the amounts of the various ingredients in the composition are given as weight percentages relative to the total weight of the composition.

To give an order of magnitude, according to the invention, the compound of formula (I) or a salt thereof, or a mixture of compounds of formula (I) and/or a salt thereof, may be used in an amount representing from 10-3% to 10% of the total weight of the composition, preferably in an amount representing from 10-3% to 5% and better still from 10-2% to 2% of the total weight of the composition, for example from 0.5 to 2%.

The composition of the invention may be for cosmetic or pharmaceutical use. The composition of the invention is preferably for cosmetic use. Thus, the composition must contain a non-toxic, physiologically acceptable medium that can be applied to human skin, including the scalp and the eyelids and to keratin fibres. For the purposes of the invention, the term “cosmetic” means a composition of pleasant appearance, odour and feel.

The compound of formula (I) or a salt thereof may be used in a composition that should be ingested, injected or applied to the skin or to keratin fibres (to any area of skin or fibres to be treated).

According to the invention, the compound of formula (I) or a salt thereof may be used orally in an amount of from 0.1 to 300 mg per day, for example from 5 to 10 mg/day.

A preferred composition of the invention is a composition for cosmetic use and in particular for topical application to the skin and keratin fibres, and more especially to the scalp, the hair and the eyelashes.

This composition may be in any known presentation form that is suitable for the mode of use.

For topical application to the skin, the composition may be in the form of an aqueous, alcoholic or aqueous-alcoholic solution or suspension, or an oily suspension, an emulsion of more or less fluid consistency and especially of liquid or semi-liquid consistency, obtained by dispersion of a fatty phase in an aqueous phase (O/W) or conversely (W/O), a solid (O/W) or (W/O) emulsion, a more or less fluid or solid aqueous, aqueous-alcoholic or oily gel, a free or compacted powder to be used in unmodified form or to be incorporated into a physiologically acceptable medium, or alternatively microcapsules, microparticles or vesicular dispersions of ionic and/or nonionic type. It may thus be in the form of a lotion, a serum, a milk, an O/W or W/O cream, an ointment, pomade, a balm, a patch or an impregnated pad.

A composition in the form of a foam or alternatively in the form of an aerosol or spray, then comprising a pressurized propellant, may also be envisaged.

In particular, the composition for application to the scalp or the hair may be in the form of a haircare lotion, for example for daily or twice-weekly application, a shampoo or a hair conditioner, in particular for twice-weekly or weekly application, a liquid or solid scalp cleansing soap for daily application, a hairstyle shaping product (lacquer, hair setting product or styling gel), a treatment mask, a foaming gel or cream for cleansing the hair. It may also be in the form of a hair dye or mascara to be applied with a brush or a comb.

Moreover, for topical application to the eyelashes and body hairs, the composition to which the invention applies may be in the form of a pigmented or unpigmented mascara, to be applied with a brush to the eyelashes or alternatively to beard or moustache hair.

For a composition for use by injection, the composition may be in the form of an aqueous lotion or an oily suspension. For oral use, the composition may be in the form of capsules, granules, drinkable syrups or tablets.

According to one particular embodiment, the composition according to the invention is in the form of a hair cream or hair lotion, or a shampoo or conditioner for the hair or for the eyelashes.

The amounts of the various constituents of the composition according to the invention are those generally used in the fields under consideration. In addition, these compositions are prepared according to the usual methods.

When the composition is an emulsion, the proportion of the fatty phase may range from 2% to 80% by weight and preferably from 5% to 50% by weight relative to the total weight of the composition. The aqueous phase is adjusted as a function of the content of fatty phase and of compound(s) (I) and also of that of the optional additional ingredients, to obtain 100% by weight. In practice, the aqueous phase represents from 5% to 99.9% by weight of the total weight of the composition.

The fatty phase may contain fatty or oily compounds that are liquid at room temperature (25° C.) and atmospheric pressure (760 mm/Hg), which are generally known as oils. These oils may be mutually compatible or incompatible and may form a macroscopically homogeneous liquid fatty phase or a two-phase or three-phase system.

In addition to the oils, the fatty phase may contain waxes, gums, lipophilic polymers or “pasty” or viscous products containing solid parts and liquid parts.

The aqueous phase contains water and optionally an ingredient that is miscible in all proportions with water, for instance C₁ to C₈ lower alcohols such as ethanol or isopropanol, polyols, for instance propylene glycol, glycerol or sorbitol, or alternatively acetone or ether.

The emulsifiers and co-emulsifiers used to obtain a composition in emulsion form are those generally used in cosmetics and pharmaceuticals. Their nature also depends on the sense of the emulsion. In practice, the emulsifier and, where appropriate, the co-emulsifier are present in the composition in a proportion ranging from 0.1% to 30% by weight, preferably from 0.5 to 20% by weight and better still from 1% to 8% by weight. The emulsion may also contain lipid vesicles and especially liposomes.

When the composition is in the form of an oily solution or gel, the fatty phase may represent more than 90% of the total weight of the composition.

Advantageously, for a hair application, the composition is an aqueous, alcoholic or aqueous-alcoholic solution or suspension and better still a water/ethanol solution or suspension. The alcoholic fraction may represent from 5% to 99.9% and especially from 8% to 80%.

For a mascara application, the composition is a wax-in-water or wax-in-oil dispersion, a gelled oil or an aqueous gel, which may be pigmented or unpigmented.

The composition of the invention may also comprise other ingredients usually used in the fields under consideration, chosen from aqueous-phase or oily-phase solvents, thickeners or gelling agents, dyes that are soluble in the medium of the composition, solid particles such as fillers or pigments, antioxidants, preserving agents, fragrances, electrolytes, neutralizers, film-forming polymers, UV blockers, for instance sunscreens, cosmetic and pharmaceutical active agents with a beneficial effect on the skin or keratin fibres, other than the compounds of formula (I), and mixtures thereof. These additives may be present in the composition in the amounts generally used in cosmetics and dermatology, and especially in a proportion of from 0.01% to 50% and better still from 0.1% to 20%, for example from 0.1% to 10%, relative to the total weight of the composition.

Needless to say, a person skilled in the art will take care to select the optional additional additives and/or the amount thereof such that the advantageous properties of the composition according to the invention, i.e. the inhibition of 15-PGDH in particular, or the increase in the density of keratin fibres (hair fibres or eyelashes), are not, or are not substantially, adversely affected by the envisaged addition.

As solvents that may be used in the invention, mention may be made of C₂ to C₈ lower alcohols, for instance ethanol, isopropanol, propylene glycol and certain light cosmetic oils, for instance C₆ to C₁₀ alkanes.

As oils that may be used in the invention, mention may be made of oils of mineral origin (liquid petroleum jelly or hydrogenated isoparaffin), oils of plant origin (liquid fraction of shea butter, sunflower oil, apricot oil, fatty alcohol or fatty acid), oils of animal origin (perhydrosqualene), synthetic oils (fatty acid ester, purcellin oil), silicone oils (linear or cyclic polydimethylsiloxane, or phenyl trimethicone) and fluoro oils (perfluoropolyethers). Waxes that may be mentioned include silicone waxes, rice wax, candelilla wax, beeswax, carnauba wax, paraffin wax and polyethylene wax.

As emulsifiers that may be used in the invention, examples that may be mentioned include glyceryl stearate, glyceryl laurate, sorbitol stearate, sorbitol oleate, alkyl dimethicone copolyols (with alkyl>8) and mixtures thereof for a W/O emulsion. Polyethylene glycol monostearate or monolaurate, polyoxyethylenated sorbitol stearate or oleate, and dimethicone copolyols, and mixtures thereof, may also be used for an O/W emulsion.

As hydrophilic gelling agents that may be used in the invention, mention may be made of carboxylvinyl polymers (carbomer), acrylic copolymers such as acrylate/alkylacrylate copolymers, polyacrylamides, polysaccharides such as hydroxypropylcellulose, natural gums and clays, and, as lipophilic gelling agents that may be used in the invention, mention may be made of modified clays, for instance Bentones, metal salts of fatty acids, for instance aluminium stearates, hydrophobic-treated silica and ethylcellulose, and mixtures thereof.

The composition may also contain a cosmetic or pharmaceutical active agent other than the compounds of formula (I), which may be hydrophilic and chosen from proteins, protein hydrolysates, amino acids, polyols, urea, allantoin, sugars and sugar derivatives, water-soluble vitamins, plant extracts (those from Iridacea plants or from soybean) and hydroxy acids (fruit acids or salicylic acid); or lipophilic and chosen from retinol (vitamin A) and its derivatives, especially an ester (retinyl palmitate), tocopherol (vitamin E) and its derivatives (tocopheryl acetate), essential fatty acids, ceramides, essential oils, salicylic acid derivatives, for instance 5-n-octanoyl salicylic acid, hydroxy acid esters, and phospholipids, for instance lecithin, and mixtures thereof.

According to one particular embodiment of the invention, the compound of formula (I) or a salt thereof may be combined with at least one additional active agent that promotes the regrowth and/or limits the loss of keratin fibres (hair or eyelashes). These additional compounds are chosen especially from the lipoxygenase inhibitors as described in EP 0 648 488, the bradykinin inhibitors described especially in EP 0 845 700, prostaglandins and derivatives thereof, especially those described in WO 98/33497, WO 95/11003, JP 97-100 091 and JP 96-134 242, prostaglandin receptor agonists or antagonists, the non-prostanoic prostaglandin analogues as described in EP 1 175 891, EP 1 175 890, WO 01/74307, WO 01/74313, WO 01/74314, WO 01/74315 or WO 01/72268, and mixtures thereof.

As other additional active compounds that promote the growth of keratin fibres (especially the hair) and/or that limit their loss, which may be present in the composition according to the invention, mention may be made of vasodilators, antiandrogens, cyclosporins and analogues thereof, antimicrobial and antifungal agents, anti-inflammatory agents, and retinoids, and mixtures thereof.

The vasodilators that may be used are especially potassium-channel agonists, including Minoxidil, and also the compounds described in patents U.S. Pat. Nos. 3,382,247, 5,756,092, 5,772,990, 5,760,043, 5,466,694, 5,438,058 and 4,973,474, cromakalim, nicorandil and diaxozide, alone or in combination.

The antiandrogens that may be used especially include steroidal and non-steroidal 5a-reductase inhibitors, for instance finasteride and the compounds described in U.S. Pat. No. 5,516,779, cyprosterone acetate, azelaic acid and the salts and derivatives thereof, and the compounds described in U.S. Pat. No. 5,480,913, flutamide, oxendolone, spironolactone, diethylstilbestrol and the compounds described in patents U.S. Pat. Nos. 5,411,981, 5,565,467 and 4,910,226.

The antimicrobial or antifungal compounds may be chosen from selenium derivatives, octopirox, ketoconazole, triclocarban, triclosan, zinc pyrithione, itraconazole, asiatic acid, hinokitiol, mipirocine, tetracyclines, especially erythromycin and the compounds described in EP 0 680 745, clinycin hydrochloride, benzoyl peroxide or benzyl peroxide, minocycline and compounds belonging to the imidazole class, such as econazole, ketoconazole or miconazole or salts thereof, nicotinic acid esters, especially including tocopheryl nicotinate, benzyl nicotinate and C₁-C₆ alkyl nicotinates, for instance methyl or hexyl nicotinate.

The anti-inflammatory agents may be chosen from steroidal anti-inflammatory agents, for instance glucocorticoids, corticosteroids (for example: hydrocortisone) and non-steroidal anti-inflammatory agents, for instance glycyrrhetinic acid and a-bisabolol, benzydamine, salicylic acid and the compounds described in EP 0 770 399, WO 94/06434 and FR 2 268 523.

The retinoids may be chosen from isotretinoin, acitretin and tazarotene.

As other active compounds for promoting the growth and/or limiting the loss of keratin fibres (especially the hair) that may be used in combination with the compound of formula (I), mention may be made of aminexil, 6-O-[(9Z,12Z)octadeca-9,12-dienoyl]hexopyranose, benzalkonium chloride, benzethonium chloride, phenol, oestradiol, chlorpheniramine maleate, chlorophylline derivatives, cholesterol, cysteine, methionine, menthol, peppermint oil, calcium pantothenate, panthenol, resorcinol, protein kinase C activators, glycosidase inhibitors, glycosaminoglycanase inhibitors, pyroglutamic acid esters, hexosaccharide or acylhexosaccharide acids, substituted aryl ethylenes, N-acylamino acids, flavonoids, ascomycin derivatives and analogues, histamine antagonists, saponins, proteoglycanase inhibitors, oestrogen agonists and antagonists, pseudoterines, cytokines, growth factor promoters, IL-1 or IL-6 inhibitors, IL-10 promoters, TNF inhibitors, benzophenones, hydantoin, retinoic acid; vitamins, for instance vitamin D, vitamin B12 analogues and pantothenol; antipruriginous agents, for instance thenaldine, trimeprazine or cyproheptadine; antiparasitic agents, in particular metronidazole, crotamiton or pyrethroids; calcium antagonists, for instance cinnarizine, diltiazem, nimodipine, verapamil and nifedipine; hormones such as oestriol or its analogues, thyroxine and its salts, and progesterone; triterpenes, for instance ursolic acid and the compounds described in U.S. Pat. No. 5,529,769, U.S. Pat. No. 5,468,888 and U.S. Pat. No. 5,631,282; FP receptor (type-F prostaglandin receptor) agonists such as latonoprost, bimatoprost, travoprost or unoprostone; mixtures thereof.

Advantageously, the composition according to the invention will comprise at least one 15-PGDH inhibitor as defined above and at least one prostaglandin or prostaglandin derivative, for instance the prostaglandins of series 2 especially including PGF2-α and PGE2 in salt or ester form (for example the isopropyl esters), derivatives thereof, for instance 16,16-dimethyl PGE2, 17-phenyl PGE2, 16,16-dimethyl PGF2-α, 17-phenyl PGF2-α, prostaglandins of series 1, for instance 11-deoxyprostaglandin E1, 1-deoxyprostaglandin E1 in salt or ester form, analogues thereof, especially latanoprost, travoprost, bimatoprost, fluprostenol, cloprostenol, viprostol, butaprost, misoprostol, unoprostone, and the salts or esters thereof.

The composition preferably contains at least one non-prosanoic EP2 and/or EP4 receptor agonist as described especially in EP 1 175 892.

It may also be envisaged for the composition comprising at least the compound of formula (I), or a salt thereof, to be in liposomal form, as described especially in document WO 94/22468. Thus, the compound encapsulated in the liposomes may be delivered selectively to the hair follicle.

The composition according to the invention may be applied to the alopecic areas of the scalp and the hair of an individual, and optionally left in contact for several hours and optionally rinsed out.

The composition containing an effective amount of a compound of formula (I) or a salt thereof may, for example, be applied in the evening, kept in contact throughout the night and optionally shampooed out in the morning. These applications may be repeated daily for one or more months according to the individual.

Advantageously, in the process according to the invention, between 5 and 500 μl of a solution or composition as defined above, comprising from 0.001% to 5% of 15-PGDH inhibitor, is applied to the areas of the scalp to be treated.

### EXAMPLES

Examples of implementation of the invention, which cannot in any way limit its scope, will now be given for illustrative purposes.

As examples of pyrazole compounds of formula (I) that may be used in the invention, mention may be made of the following compounds:

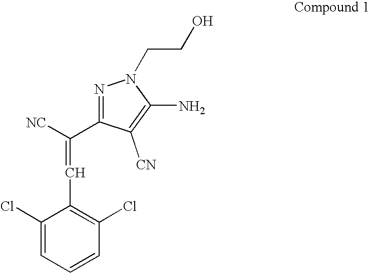

and more especially compound 1a (ring in the Z position of the double bond)

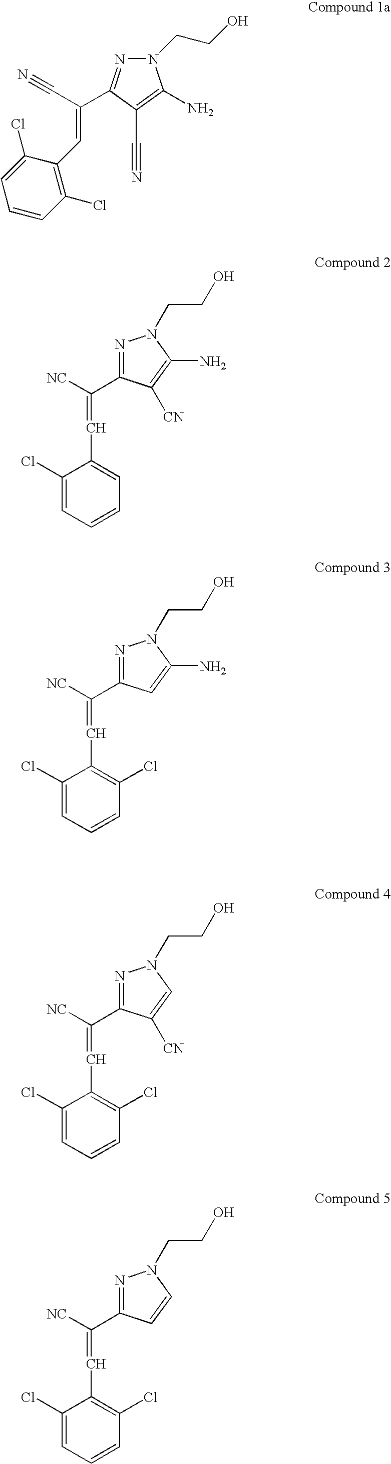

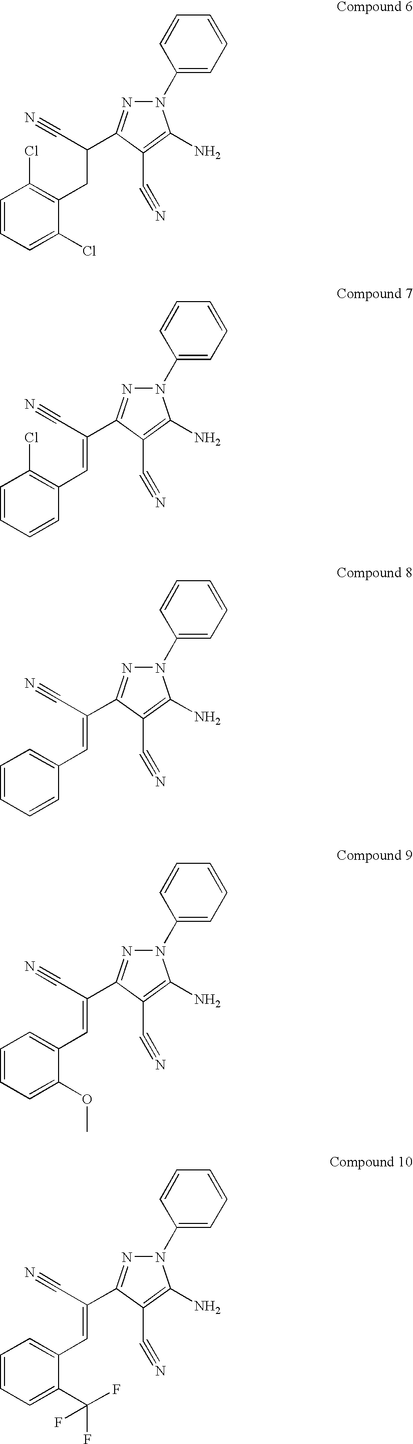

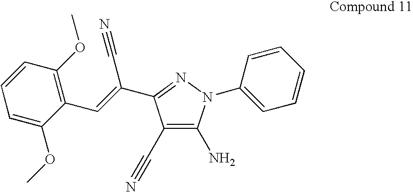

Compound 11 may be mentioned as a novel pyrazole compound of formula (I) or (III).

### Example 1

### Procedure for the synthesis of 5-amino-3-[1-cyano-2-(2,6-dimethoxyphenyl)vinyl]-1-phenyl-1H-pyrazole-4-carbonitrile (Compound 11).

1 g (4.48 mmol) of 5-amino-4-cyano-1-phenyl-3-pyrazoleacetonitrile is suspended in 15 ml of toluene in a round-bottomed flask under an argon atmosphere, on which is mounted Dean-Stark apparatus. 0.744 g (1 eq.) of 2,6-dimethoxybenzaldehyde and 0.030 ml of piperidine are added to the mixture. The reaction mixture is refluxed overnight and then allowed to cool to room temperature. A whitish precipitate forms and is filtered off and washed with toluene. The filtrate is concentrated to dryness and the residue is taken up in ethanol with stirring for 15 minutes. The suspension is filtered and the filtrate is concentrated to dryness. The residue is combined with the precipitate obtained previously, and purified on silica gel (eluant: 98/2 dichloromethane/methanol). 619 mg of product are thus obtained in a yield of 37%.

Analyses:

Mass spectrometry: (ESI±in CH₃OH/H₂O): 372(MH)⁺, 394(MNa)⁺, 743(2MH)⁺, 765(2MNa)⁺, 370(M-H) Nuclear Magnetic Resonance: ¹H (400 MHz; DMSO-d₆) δppm: 3.85 (s, 6H, OCH₃(13) and OCH₃(9)); 6.78 (d, 2H, H(10) and H(12)); 6.95 (s, 2H, NH₂(3)); 7.42 to 7.57 (m, 6H, H(2′) to H(6′) and H(11)); 7.86 (s, 1H, H(7)).

Elemental Analysis:

| | | | | | | | | |
|---|---|---|---|---|---|---|---|---|
| Theory: | C | 67.91%; | H | 4.61% | N | 18.86%; | O | 8.62% |
| Analysis: | C | 67.30%; | H | 4.46%; | N | 18.88%; | O | 8.96% |

### Example 2

### Demonstration of the 15-PGDH-Specific Inhibitory Properties of the Compounds of Formula (1). 1) Test on 15-PGDH

The enzyme 15-PGDH is obtained as described in patent application FR 02/05067 filed in the name of L'Oreal, as a suspension in a medium adjusted to a concentration of 0.3 mg/ml and then blocked at −80° C. For the purposes of the test, this suspension is thawed and stored in ice.

In parallel, a 100 mM, pH 7.4 Tris buffer containing 0.1 mM of dithiothreitol (D5545, Sigma-Aldrich, L'isle D'Abeau Chesne, BP 701, 38297, Saint Quentin Fallavier), 1.5 mM of P-NAD (N6522, Sigma-Aldrich, L'isle D'Abeau Chesne, BP 701, 38297, Saint Quentin Fallavier), and 50 μM of prostaglandin E₂ (P4172, Sigma-Aldrich, L'isle D'Abeau Chesne, BP 701, 38297, Saint Quentin Fallavier) is prepared.

0.965 ml of this buffer (brought to 37° C. beforehand) is introduced into the cuvette of a spectrophotometer (Perkin-Elmer, Lambda 2) thermostatically maintained at 37° C., the measuring wavelength of which is set at 340 nm. 0.035 ml of enzymatic suspension at 37° C. is introduced into the cuvette concomitantly with the recording (corresponding to an increase in the optical density at 340 nm). The maximum reaction rate is recorded.

The test values (containing the compounds (I)) are compared with the control value (without compound (I)); the results indicated represent the concentration at which compound (I) inhibits 50% of the enzymatic activity of 15-PGDH, noted as IC_{50dh}.

2) Test on PGF Synthase

The enzyme PGFS is obtained as described in document FR-A-02/05067, at a concentration of 0.5 mg/ml, as a suspension in a suitable medium, and blocked at −80° C. For the purposes of the test, this suspension is thawed and stored in ice.

In parallel, a 100 mM, pH 6.5 Tris buffer containing 20 μm of 9,10-phenanthrenequinone* (P2896, Sigma-Aldrich, L'isle D'Abeau Chesne, BP 701, 38297, Saint Quentin Fallavier) and 100 μM of β-NADPH (N1630, Sigma-Aldrich, L'isle D'Abeau Chesne, BP 701, 38297, Saint Quentin Fallavier) is prepared in a brown flask (protected from light).

*A stock solution with a titre of 1 mM is prepared in absolute ethanol and brought to 40° C.; the flask is placed in an ultrasound tank to facilitate the dissolution of the product.

0.950 ml of this buffer (brought to 37° C. beforehand) is introduced into the cuvette of a spectrophotometer (Perkin-Elmer, Lambda 2) thermostatically maintained at 37° C., the measuring wavelength of which is set at 340 nm. 0.05 ml of enzymatic suspension at 37° C. is introduced into the cuvette concomitantly with the recording (corresponding to a reduction in the optical density at 340 nm). The maximum reaction rate is recorded.

The test values (containing compound (I)) are compared with the control value (without compound (I)); the results indicated represent the concentration at which compound (I) inhibits 50% of the enzymatic activity of PGFS, noted as IC_{50fs}.

| | | | Inhibition | |
|---|---|---|---|---|
| | | Inhibition | of PGF | |
| | | of 15-PGDH | synthase | |
| Compound | Structure | IC_{50dh} μM | IC_{50fs} μM | Selectivity |
| 1a | 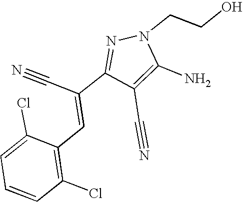 | 3 | >50 | >16.6 |
| 6 | 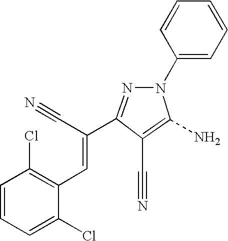 | 0.8 | >50 | >62 |
| 7 | 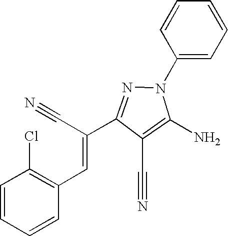 | 3 | >50 | >16 |
| 8 | 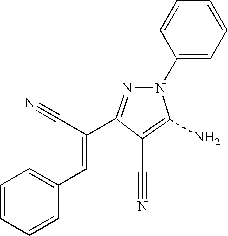 | 50 | >75 | >1.5 |
| 9 | 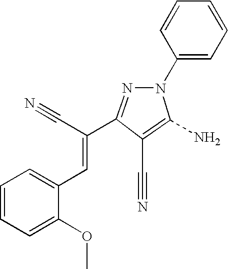 | 5 | >50 | >10 |

It emerges from this table that the IC_{50fs}/IC_{50dh} ratio of compounds 1a, 6, 7, 8 and 9 is >1.5. compounds la, 6, 7, 8 and 9, and more especially 1a, 6, 7 and 9, thus show selective inhibitory activity towards 15-PGDH relative to PGF synthase.

The compositions below are obtained via the usual techniques commonly used in cosmetics or pharmaceutics.

### Example 3

### Hair Lotion

| | | |
|---|---|---|
| Compound 1a | 0.80 | g |
| Propylene glycol | 10.00 | g |
| Isopropyl alcohol | qs 100.00 | g |

This lotion is applied to the scalp, once or twice a day, at a rate of 1 ml per application, massaging the scalp gently to help the active agent to penetrate. The head of hair is then dried in the open air. This lotion makes it possible to reduce hair loss and to promote regrowth of the hair.

### Example 4

### Hair Lotion

| | | |
|---|---|---|
| Compound 2 | 1.00 | g |
| Propylene glycol | 30.00 | g |
| Ethyl alcohol | 40.00 | g |
| Water | qs 100.00 | g |

This lotion is applied to the scalp, once or twice a day, at a rate of 1 ml per application, massaging the scalp gently to help the active agent to penetrate. The head of hair is then dried in the open air.

### Example 5

### Hair Lotion

| | | |
|---|---|---|
| Compound 1a | 1 | g |
| Ethyl alcohol | 40.00 | g |
| HCl | qs (*) | |
| Water | qs 100.00 | g |

(*) Quantity sufficient to neutralize the amine function borne on the pyrazole nucleus.

This lotion is applied to the scalp, once or twice a day, at a rate of 1 ml per application, massaging the scalp gently to help the active agent to penetrate.

### Example 6

### Hair Lotion

| | | |
|---|---|---|
| Compound 1a | 0.10 | g |
| Latanoprost | 0.10 | g |
| Propylene glycol | 30.00 | g |
| Ethyl alcohol | 40.00 | g |
| Water | qs 100.00 | g |

### Example 7

### Wax/Water Mascara

| | |
|---|---|
| Beeswax | 6.00% |
| Paraffin wax | 13.00% |
| Hydrogenated jojoba oil | 2.00% |
| Water-soluble film-forming polymer | 3.00% |
| Triethanolamine stearate | 8.00% |
| Compound 5 | 1.00% |
| Black pigment | 5.00% |
| Preserving agent | qs |
| Water | qs |
| | 100.00% |

This mascara is applied to the eyelashes like a standard mascara with a mascara brush.

1. Use of an effective amount of at least one styrylpyrazole compound of formula (I), or a salt thereof: 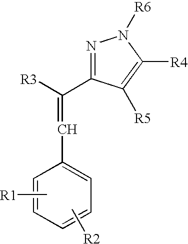
in which:
R₁, R₂, R₄ and R₅, which may be identical or different, are chosen from hydrogen, a halogen, groups OR₇, SR₇, NR₇R′₇, COOR₇, CONR₇R′₇, CF₃, CN, NR₇COR′₇, SO₂R₇, SO₂NR₇R′₇, NR₇SO₂R′₇, COR₇, CSR₇, OCOR₇, COSR₇, SCOR₇, CSNR₇R′₇, NR₇CONR′₇R″₇, NR₇C(═NR′₇)NR″₇R″′₇, NR₇CSR′₇ and NR₇CSNR′₇R″₇, saturated or unsaturated, linear or branched C₁-C₂₀ alkyl radicals, saturated or unsaturated rings of 4 to 7 atoms, optionally containing at least one hetero atom, these rings possibly being separate or fused, the alkyl radicals and the rings also possibly being substituted with at least one substituent A₁, with R₇, R′₇, R″₇ and R″′₇ independently denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₂;
R₃ is chosen from CN, COOR₈, CONR₈R′₈, COR₈, SO₂R₈ and SO₂NR₈R′₈, with R₈ and R′₈ independently denoting hydrogen, a linear or branched C₁-C₂₀ alkyl radical or a ring of 4 to 7 atoms, isolated or fused to another ring and optionally containing at least one hetero atom, the alkyl radical or the said rings being saturated or unsaturated and optionally substituted with at least one substituent A₃;
R₆ is chosen from hydrogen, groups COOR₉, COR₉, CSR₉, COSR₉, CONR₉R′₉, SO₂R₉ and SO₂NR₉R′₉, linear or branched, saturated or unsaturated C₁-C₂₀ alkyl radicals and saturated or unsaturated rings of 4 to 7 atoms, optionally containing at least one hetero atom, these rings possibly being separate or fused, the alkyl radicals and the rings also possibly being substituted with at least one substituent A₄, with R₉ and R′₉, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₅;
A₁, A₂, A₃, A₄ and A₅ being chosen independently from halogens, groups OR₁₀, SR₁₀, NR₁₀R′₁₀, COOR₁₀, CH₂COOR₁₀, CONR₁₀R′₁₀, CF₃, CN, NR₁₀COR′₁₀, SO₂R₁₀, SO₂NR₁₀R′₁₀, NR₁₀SO₂R′₁₀, COR₁₀, CSR₁₀, OCOR₁₀, COSR₁₀, SCOR₁₀, CSNR₁₀R′₁₀, NR₁₀CONR′₁₀R″₁₀, NR₁₀C(═NR′₁₀)NR″₁₀R″₁₀, NR₁₀CSNR+₁₀R″₁₀ and NR₁₀CSR′₁₀, with R₁₀, R′₁₀, R″₁₀ and R″′₁₀, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated,
as an agent for inducing and/or stimulating the growth of keratin fibres, especially human keratin fibres, and/or for reducing their loss and/or increasing their density.

2. Cosmetic use of at least one styrylpyrazole compound of formula (I), or a salt thereof: 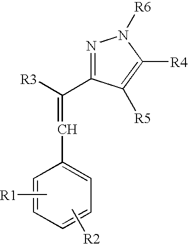
in which:
R₁, R₂, R₄ and R₅, which may be identical or different, are chosen from hydrogen, a halogen, groups OR₇, SR₇, NR₇R′₇, COOR₇, CONR₇R′₇, CF₃, CN, NR₇COR′₇, SO₂R₇, SO₂NR₇R′₇, NR₇SO₂R′₇, COR₇, CSR₇, OCOR₇, COSR₇, SCOR₇, CSNR₇R′₇, NR₇CONR′₇R″₇, NR₇C(═NR′₇)NR″₇R″′₇, NR₇CSR′₇ and NR₇CSNR′₇R″₇, saturated or unsaturated, linear or branched C₁-C₂₀ alkyl radicals, saturated or unsaturated rings of 4 to 7 atoms, optionally containing at least one hetero atom, these rings possibly being separate or fused, the alkyl radicals and the rings also possibly being substituted with at least one substituent A₁, with R₇, R′₇, R″₇ and R″′₇ independently denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₂;
R₃ is chosen from CN, COOR₈, CONR₈R′₈, COR₈, SO₂R₈ and SO₂NR₈R′₈, with R₈ and R′₈ independently denoting hydrogen, a linear or branched C₁-C₂₀ alkyl radical or a ring of 4 to 7 atoms, isolated or fused to another ring and optionally containing at least one hetero atom, the alkyl radical or the said rings being saturated or unsaturated and optionally substituted with at least one substituent A₃;
R₆ is chosen from hydrogen, groups COOR₉, COR₉, CSR₉, COSR₉, CONR₉R′₉, SO₂R₉ and SO₂NR₉R′₉, linear or branched, saturated or unsaturated C₁-C₂₀ alkyl radicals and saturated or unsaturated rings of 4 to 7 atoms, optionally containing at least one hetero atom, these rings possibly being separate or fused, the alkyl radicals and the rings also possibly being substituted with at least one substituent A₄, with R₉ and R′₉ which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₅;
A₁, A₂, A₃, A₄ and A₅ being chosen independently from halogens, groups OR₁₀, SR₁₀, NR₁₀R′₁₀, COOR₁₀, CH₂COOR₁₀, CONR₁₀R′₁₀, CF₃, CN, NR₁₀COR′₁₀, SO₂R₁₀, SO₂NR₁₀R′₁₀, NR₁₀SO₂R′₁₀, COR₁₀, CSR₁₀, OCOR₁₀, COSR₁₀, SCOR₁₀, CSNR₁₀R′₁₀, NR₁₀CONR′₁₀R″₁₀, NR₁₀C(═NR′₁₀)NR″₁₀R″′₁₀, NR₁₀CSNR′₁₀R″₁₀ and NR₁₀CSR′₁₀, with R₁₀, R′₁₀, R″₁₀ and R″′₁₀, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated,
in a cosmetic care and/or makeup composition for human keratin fibres, to induce and/or stimulate their growth, reduce their loss and/or increase their density.

3. Use of at least one styrylpyrazole compound of formula (I), or a salt thereof: 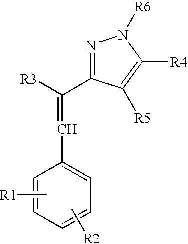
in which:
R₁, R₂, R₄ and R₅, which may be identical or different, are chosen from hydrogen, a halogen, groups OR₇, SR₇, NR₇R′₇, COOR₇, CONR₇R′₇, CF₃, CN, NR₇COR′₇, SO₂R₇, SO₂NR₇R′₇, NR₇SO₂R′₇, COR₇, CSR₇, OCOR₇, COSR₇, SCOR₇, CSNR₇R′₇, NR₇CONR′₇R″₇, NR₇C(═NR′₇)NR″₇R″′₇, NR₇CSR′₇ and NR₇CSNR′₇R″₇, saturated or unsaturated, linear or branched C₁-C₂₀ alkyl radicals, saturated or unsaturated rings of 4 to 7 atoms, optionally containing at least one hetero atom, these rings possibly being separate or fused, the alkyl radicals and the rings also possibly being substituted with at least one substituent Al, with R₇, R′₇, R″₇ and R″′₇ independently denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₂;
R₃ is chosen from CN, COOR₈, CONR₈R′₈, COR₈, SO₂R₈ and SO₂NR₈R′₈, with R₈ and R′₈ independently denoting hydrogen, a linear or branched C₁-C₂₀ alkyl radical or a ring of 4 to 7 atoms, isolated or fused to another ring and optionally containing at least one hetero atom, the alkyl radical or the said rings being saturated or unsaturated and optionally substituted with at least one substituent A₃;
R₆ is chosen from hydrogen, groups COOR₉, COR₉, CSR₉, COSR₉, CONR₉R′₉, SO₂R₉ and SO₂NR₉R′₉, linear or branched, saturated or unsaturated C₁-C₂₀ alkyl radicals and saturated or unsaturated rings of 4 to 7 atoms, optionally containing at least one hetero atom, these rings possibly being separate or fused, the alkyl radicals and the rings also possibly being substituted with at least one substituent A₄, with R₉ and R′₉, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₅;
A₁, A₂, A₃, A₄ and A₅ being chosen independently from halogens, groups OR₁₀, SR₁₀, NR₁₀R′₁₀, COOR₁₀, CH₂COOR₁₀, CONR₁₀R′₁₀, CF₃, CN, NR₁₀COR′₁₀, SO₂R₁₀, SO₂NR₁₀R′₁₀, NR₁₀SO₂R′₁₀, COR₁₀, CSR₁₀, OCOR₁₀, COSR₁₀, SCOR₁₀, CSNR₁₀R′₁₀, NR₁₀CONR′₁₀R″₁₀, NR₁₀C(═NR′₁₀)NR″₁₀R″₁₀, NR₁₀CSNR′₁₀R″₁₀ and NR₁₀CSR′₁₀, with R₁₀, R′₁₀, R″₁₀ and R″′₁₀, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated,
for the preparation of a care or treatment composition for human keratin fibres, which is intended to induce and/or stimulate the growth of the said fibres and/or reduce their loss and/or increase their density.

4. Use of at least one styrylpyrazole compound of formula (I), or a salt thereof: 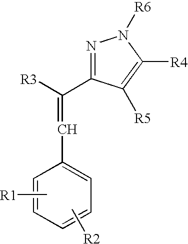
in which:
R₁, R₂, R₄ and R₅, which may be identical or different, are chosen from hydrogen, a halogen, groups OR₇, SR₇, NR₇R′₇, COOR₇, CONR₇R′₇, CF₃, CN, NR₇COR′₇, SO₂R₇, SO₂NR₇R′₇, NR₇SO₂R′₇, COR₇, CSR₇, OCOR₇, COSR₇, SCOR₇, CSNR₇R′₇, NR₇CONR′₇R″₇, NR₇C(═NR′₇)NR″₇R″′₇, NR₇CSR′₇ and NR₇CSNR′₇R″₇, saturated or unsaturated, linear or branched C₁-C₂₀ alkyl radicals, saturated or unsaturated rings of 4 to 7 atoms, optionally containing at least one hetero atom, these rings possibly being separate or fused, the alkyl radicals and the rings also possibly being substituted with at least one substituent A₁, with R₇, R′₇, R″₇ and R″′₇ independently denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₂;
R₃ is chosen from CN, COOR₈, CONR₈R′₈, COR₈, SO₂R₈ and SO₂NR₈R′₈, with R₈ and R′₈ independently denoting hydrogen, a linear or branched C₁-C₂₀ alkyl radical or a ring of 4 to 7 atoms, isolated or fused to another ring and optionally containing at least one hetero atom, the alkyl radical or the said rings being saturated or unsaturated and optionally substituted with at least one substituent A₃;
R₆ is chosen from hydrogen, groups COOR₉, COR₉, CSR₉, COSR₉, CONR₉R′₉, SO₂R₉ and SO₂NR₉R′₉, linear or branched, saturated or unsaturated C₁-C₂₀ alkyl radicals and saturated or unsaturated rings of 4 to 7 atoms, optionally containing at least one hetero atom, these rings possibly being separate or fused, the alkyl radicals and the rings also possibly being substituted with at least one substituent A₄, with R₉ and R′₉, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₅;
A₁, A₂, A₃, A₄ and A₅ being chosen independently from halogens, groups OR₁₀, SR₁₀, NR₁₀R′₁₀, COOR₁₀, CH₂COOR₁₀, CONR₁₀R′₁₀, CF₃, CN, NR₁₀COR′₁₀SO₂R₁₀, SO₂NR₁₀R′₁₀, NR₁₀SO₂R′₁₀, COR₁₀, CSR₁₀, OCOR₁₀, COSR₁₀, SCOR₁₀, CSNR₁₀R′₁₀, NR₁₀CONR′₁₀R″₁₀, NR₁₀C (═NR′₁₀)NR″₁₀R″′₁₀, NR₁₀CSNR′₁₀R″₁₀ and NR₁₀CSR′₁₀, with R₁₀, R′₁₀, R″₁₀ and R″′₁₀, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated,
as an inhibitor of 15-hydroxyprostaglandin dehydrogenase, especially human 15-hydroxyprostaglandin dehydrogenase.

5. Use of at least one styrylpyrazole compound of formula (I), or a salt thereof: 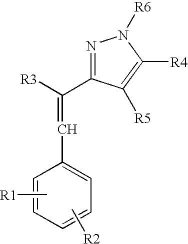
in which:
R₁, R₂, R₄ and R₅, which may be identical or different, are chosen from hydrogen, a halogen, groups OR₇, SR₇, NR₇R′₇, COOR₇, CONR₇R′₇, CF₃, CN, NR₇COR′₇, SO₂R₇, SO₂NR₇R′₇, NR₇SO₂R′₇, COR₇, CSR₇, OCOR₇, COSR₇, SCOR₇, CSNR₇R′₇, NR₇CONR′₇R″₇, NR₇C(═NR′₇)NR″₇R″′₇, NR₇CSR′₇ and NR₇CSNR′₇R″₇, saturated or unsaturated, linear or branched C₁-C₂₀ alkyl radicals, saturated or unsaturated rings of 4 to 7 atoms, optionally containing at least one hetero atom, these rings possibly being separate or fused, the alkyl radicals and the rings also possibly being substituted with at least one substituent A₁, with R₇, R′₇, R″₇ and R″′₇ independently denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₂;
R₃ is chosen from CN, COOR8, CONR₈R′₈, COR₈, SO₂R₈ and SO₂NR₈R′₈, with R₈ and R′₈ independently denoting hydrogen, a linear or branched C₁-C₂₀ alkyl radical or a ring of 4 to 7 atoms, isolated or fused to another ring and optionally containing at least one hetero atom, the alkyl radical or the said rings being saturated or unsaturated and optionally substituted with at least one substituent A₃;
R₆ is chosen from hydrogen, groups COOR₉, COR₉, CSR₉, COSR₉, CONR₉R′₉, SO₂R₉ and SO₂NR₉R′₉, linear or branched, saturated or unsaturated C₁-C₂₀ alkyl radicals and saturated or unsaturated rings of 4 to 7 atoms, optionally containing at least one hetero atom, these rings possibly being separate or fused, the alkyl radicals and the rings also possibly being substituted with at least one substituent A₄, with R₉ and R′₉, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₅;
A₁, A₂, A₃, A₄ and A₅ being chosen independently from halogens, groups OR₁₀, SR₁₀, NR₁₀R′₁₀, COOR₁₀, CH₂COOR₁₀, CONR₁₀R′₁₀, CF₃/ CN, NR₁₀COR′₁₀, SO₂R₁₀, SO₂NR₁₀R′₁₀, NR₁₀SO₂R′₁₀, COR₁₀, CSR₁₀, OCOR₁₀, COSR₁₀, SCOR₁₀, CSNR₁₀R′10, NR₁₀CONR′₁₀R″₁₀, NR₁₀C(═NR′₁₀)NR″₁₀R″′₁₀, NR₁₀CSNR′₁₀R′₁₀ and NR₁₀CSR′₁₀, with R₁₀, R′₁₀, R″₁₀ and R′₁₀, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated,
for the manufacture of a care or treatment composition for human keratin fibres, which is intended to treat disorders associated with 15-hydroxyprostaglandin dehydrogenase in humans.

6. Use according to one of the preceding claims, characterized in that the keratin fibres are head hair, the eyebrows, the eyelashes, beard hair, moustache hair and pubic hair.

7. Use of an effective amount of at least one styrylpyrazole compound of formula (I), or a salt thereof: 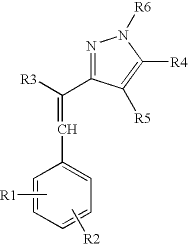
in which:
R₁, R₂, R₄ and R₅, which may be identical or different, are chosen from hydrogen, a halogen, groups OR₇, SR₇, NR₇R′₇, COOR₇, CONR₇R′₇, CF₃, CN, NR₇COR′₇, SO₂R₇, SO₂NR₇R′₇, NR₇SO₂R′₇, COR₇, CSR₇, OCOR₇, COSR₇, SCOR₇, CSNR₇R′₇, NR₇CONR′₇R″₇, NR₇C(═NR′₇)NR|₇R″′₇, NR₇CSR′₇ and NR₇CSNR′₇R″₇, saturated or unsaturated, linear or branched C₁-C₂₀ alkyl radicals, saturated or unsaturated rings of 4 to 7 atoms, optionally containing at least one hetero atom, these rings possibly being separate or fused, the alkyl radicals and the rings also possibly being substituted with at least one substituent A₁, with R₇, R′₇, R″₇ and R″′₇ independently denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₂;
R₃ is chosen from CN, COOR₈, CONR₈R′₈, COR₈, SO₂R₈ and SO₂NR₈R′₈, with R₈ and R′₈ independently denoting hydrogen, a linear or branched C₁-C₂₀ alkyl radical or a ring of 4 to 7 atoms, isolated or fused to another ring and optionally containing at least one hetero atom, the alkyl radical or the said rings being saturated or unsaturated and optionally substituted with at least one substituent A₃;
R₆ is chosen from hydrogen, groups COOR₉, COR₉, CSR₉, COSR₉, CONR₉R′₉, SO₂R₉ and SO₂NR₉R′₉, linear or branched, saturated or unsaturated C₁-C₂₀ alkyl radicals and saturated or unsaturated rings of 4 to 7 atoms, optionally containing at least one hetero atom, these rings possibly being separate or fused, the alkyl radicals and the rings also possibly being substituted with at least one substituent A₄, with R₉ and R′₉, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₅;
A₁, A₂, A₃, A₄ and A₅ being chosen independently from halogens, groups OR₁₀, SR₁₀, NR₁₀R′₁₀, COOR₁₀, CH₂COOR₁₀, CONR₁₀R′₁₀, CF₃, CN, NR₁₀COR′₁₀, SO₂R₁₀, SO₂NR₁₀R′₁₀, NR₁₀SO₂R′₁₀, COR₁₀, CSR₁₀, OCOR₁₀, COSR₁₀, SCOR₁₀, CSNR₁₀R′₁₀, NR₁₀CONR′₁₀R″₁₀, NR₁₀C(═NR′₁₀)NR″₁₀R″′₁₀, NR₁₀CSNR′₁₀R″₁₀ and NR₁₀CSR′₁₀, with R₁₀, R′₁₀, R″₁₀ and R″′₁₀, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated,
in a human cosmetic haircare composition to reduce hair loss and/or to increase its density and/or to treat alopecia of natural origin.

8. Use of at least one styrylpyrazole compound of formula (I), or a salt thereof: 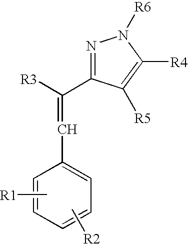
in which:
R₁, R₂, R₄ and R₅, which may be identical or different, are chosen from hydrogen, a halogen, groups OR₇, SR₇, NR₇R′₇, COOR₇, CONR₇R′₇, CF₃, CN, NR₇COR′₇, SO₂R₇, SO₂NR₇R′₇, NR₇SO₂R′₇, COR₇, CSR₇, OCOR₇, COSR₇, SCOR₇, CSNR₇R′₇, NR₇CONR′₇R″₇, NR₇C(═NR′₇)NR″₇R″′₇, NR₇CSR′₇ and NR₇CSNR′₇R″₇, saturated or unsaturated, linear or branched C₁-C₂₀ alkyl radicals, saturated or unsaturated rings of 4 to 7 atoms, optionally containing at least one hetero atom, these rings possibly being separate or fused, the alkyl radicals and the rings also possibly being substituted with at least one substituent A₁, with R₇, R′₇, R″₇ and R″′₇ independently denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₂;
R₃ is chosen from CN, COOR₈, CONR₈R′₈, COR₈, SO₂R₈ and SO₂NR₈R′₈, with R₈ and R′₈ independently denoting hydrogen, a linear or branched C₁-C₂₀ alkyl radical or a ring of 4 to 7 atoms, isolated or fused to another ring and optionally containing at least one hetero atom, the alkyl radical or the said rings being saturated or unsaturated and optionally substituted with at least one substituent A₃;
R₆ is chosen from hydrogen, groups COOR₉, COR₉, CSR₉, COSR₉, CONR₉R′₉, SO₂R₉ and SO₂NR₉R′₉, linear or branched, saturated or unsaturated C₁-C₂₀ alkyl radicals and saturated or unsaturated rings of 4 to 7 atoms, optionally containing at least one hetero atom, these rings possibly being separate or fused, the alkyl radicals and the rings also possibly being substituted with at least one substituent A₄, with R₉ and R′₉, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₅;
A₁, A₂, A₃, A₄ and A₅ being chosen independently from halogens, groups OR₁₀, SR₁₀, NR₁₀R′₁₀, COOR₁₀, CH₂COOR₁₀, CONR₁₀R′₁₀, CF₃/CN, NR₁₀COR′₁₀, SO₂R₁₀, SO₂NR₁₀R′₁₀, NR₁₀SO₂R′₁₀, COR₁₀l CSR₁₀, OCOR₁₀, COSR₁₀, SCOR₁₀, CSNR₁₀R′₁₀, NR₁₀CONR′₁₀R″₁₀, NR₁₀C(═NR′₁₀)NR″₁₀R″′₁₀, NR₁₀CSNR′₁₀OR″₁₀ and NR₁₀CSR′₁₀, with R₁₀, R′₁₀, R″₁₀ and R″′₁₀, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated,
for the preparation of a human hair composition, which is intended to induce and/or stimulate hair growth and/or reduce its loss and/or increase its density and/or treat androgenic alopecia and/or treat natural alopecia.

9. Use of at least one styrylpyrazole compound of formula (I), or a salt thereof: 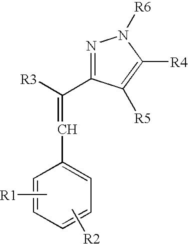
in which:
R₁, R₂, R₄ and R₅, which may be identical or different, are chosen from hydrogen, a halogen, groups OR₇, SR₇, NR₇R′₇, COOR₇, CONR₇R′₇, CF₃, CN, NR₇COR′₇, SO₂R₇, SO₂NR₇R′₇, NR₇SO₂R′₇, COR₇, CSR₇, OCOR₇, COSR₇, SCOR₇, CSNR₇R′₇, NR₇CONR′₇R″₇, NR₇C(═NR′₇)NR″₇R″′₇, NR₇CSR′₇ and NR₇CSNR′₇R″₇, saturated or unsaturated, linear or branched C₁-C₂₀ alkyl radicals, saturated or unsaturated rings of 4 to 7 atoms, optionally containing at least one hetero atom, these rings possibly being separate or fused, the alkyl radicals and the rings also possibly being substituted with at least one substituent A₁, with R₇, R′₇, R″₇ and R″′₇ independently denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₂;
R₃ is chosen from CN, COOR₈, CONR₈R′₈, COR₈, SO₂R₈ and SO₂NR₈R′₈, with R₈ and R′₈ independently denoting hydrogen, a linear or branched C₁-C₂₀ alkyl radical or a ring of 4 to 7 atoms, isolated or fused to another ring and optionally containing at least one hetero atom, the alkyl radical or the said rings being saturated or unsaturated and optionally substituted with at least one substituent A₃;
R₆ is chosen from hydrogen, groups COOR₉, COR₉, CSR₉, COSR₉, CONR₉R′₉, SO₂R₉ and SO₂NR₉R′₉, linear or branched, saturated or unsaturated C₁-C₂₀ alkyl radicals and saturated or unsaturated rings of 4 to 7 atoms, optionally containing at least one hetero atom, these rings possibly being separate or fused, the alkyl radicals and the rings also possibly being substituted with at least one substituent A₄, with R₉ and R′₉, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₅;
A₁, A₂, A₃, A₄ and A₅ being chosen independently from halogens, groups OR₁₀, SR₁₀, NR₁₀R′₁₀, COOR₁₀, CH₂COOR₁₀, CONR₁₀R′₁₀/CF₃, CN, NR₁₀COR′₁₀, SO₂R₁₀, SO₂NR₁₀R′₁₀, NR₁₀SO₂R′₁₀, COR₁₀, CSR₁₀, OCOR₁₀, COSR₁₀, SCOR₁₀, CSNR₁₀R′₁₀, NR₁₀CONR′₁₀R″₁₀, NR₁₀C(═NR′₁₀)NR″₁₀R″′₁₀, NR₁₀CSNR′₁₀R″₁₀ and NR₁₀CSR′₁₀, with R₁₀, R′₁₀, R″₁₀ and R″′₁₀, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated,
in a cosmetic care and/or makeup composition for human eyelashes, to induce and/or stimulate the growth of the eyelashes and/or to increase their density.

10. Use of at least one styrylpyrazole compound of formula (I), or a salt thereof: 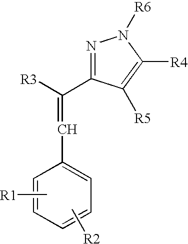
in which:
R₁, R₂, R₄ and R₅, which may be identical or different, are chosen from hydrogen, a halogen, groups OR₇, SR₇, NR₇R′₇, COOR₇, CONR₇R′₇, CF₃, CN, NR₇COR′₇, SO₂R₇, SO₂NR₇R′₇, NR₇SO₂R′₇, COR₇, CSR₇, OCOR₇, COSR₇, SCOR₇, CSNR₇R′₇, NR₇CONR′₇R″₇, NR₇C(═NR′₇)NR″₇R″′₇, NR₇CSR′₇ and NR₇CSNR′₇R″₇, saturated or unsaturated, linear or branched C₁-C₂₀ alkyl radicals, saturated or unsaturated rings of 4 to 7 atoms, optionally containing at least one hetero atom, these rings possibly being separate or fused, the alkyl radicals and the rings also possibly being substituted with at least one substituent A₁, with R₇, R′₇, R″₇ and R″′₇ independently denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₂;
R₃ is chosen from CN, COOR₈, CONR₈R′₈, COR₈, SO₂R₈ and SO₂NR₈R′₈, with R₈ and R′₈ independently denoting hydrogen, a linear or branched C₁-C₂₀ alkyl radical or a ring of 4 to 7 atoms, isolated or fused to another ring and optionally containing at least one hetero atom, the alkyl radical or the said rings being saturated or unsaturated and optionally substituted with at least one substituent A₃;
R₆ is chosen from hydrogen, groups COOR₉, COR₉, CSR₉, COSR₉, CONR₉R′₉, SO₂R₉ and SO₂NR₉R′₉, linear or branched, saturated or unsaturated C₁-C₂₀ alkyl radicals and saturated or unsaturated rings of 4 to 7 atoms, optionally containing at least one hetero atom, these rings possibly being separate or fused, the alkyl radicals and the rings also possibly being substituted with at least one substituent A₄, with R₉ and R′₉, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₅;
A₁, A₂, A₃, A₄ and A₅ being chosen independently from halogens, groups OR₁₀, SR₁₀, NR₁₀R′₁₀ COOR₁₀, CH₂COOR₁₀, CONR₁₀R′₁₀, CF₃, CN, NR₁₀COR′₁₀, SO₂R₁₀, SO₂NR₁₀R′₁₀, NR₁₀SO₂R′₁₀, COR₁₀, CSR₁₀, OCOR₁₀, COSR₁₀, SCOR₁₀, CSNR₁₀R′₁₀, NR₁₀CONR′₁₀R″₁₀, NR₁₀C(═NR′₁₀)NR″₁₀R″′₁₀, NR₁₀CSNR′₁₀R″₁₀ and NR₁₀CSR′₁₀, with R₁₀, R′₁₀, R″₁₀ and R″′₁₀, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated,
for the preparation of a care or treatment composition for human eyelashes, which is intended to induce and/or stimulate the growth of the eyelashes and/or increase their density.

11. Use according to one of the preceding claims, characterized in that the styrylpyrazole compound is of formula (II) below, or a salt thereof: 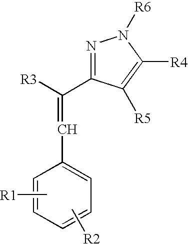
in which:
R₁, R₂, R₄ and R₅ independently represent H, a halogen, OR₇, SR₇, NR₇R′₇, COOR₇, CONR₇R′₇, CF₃, CN, a saturated or unsaturated C₁-C₁₀ alkyl radical, a saturated or unsaturated ring, separate or fused to another ring, optionally containing at least one hetero atom, the alkyl radicals and the rings also possibly being substituted with at least one substituent A₁, with R₇ and R′₇ independently denoting H, a C₁-C₁₀ alkyl radical or a ring which is isolated or fused to another ring;
R₃ represents CN, COOR₈, CONR₈R′₈ or COR₈, with R₈ and R′₈ independently denoting H, a C₁-C₁₀ alkyl radical or a ring which is isolated or fused to another ring and optionally containing at least one hetero atom, the said rings being saturated or unsaturated and optionally substituted with at least one substituent A₁;
R₆ represents hydrogen, COOR₉, COR₉, a saturated or unsaturated C₁-C₁₀ alkyl radical or a saturated or unsaturated ring, which is separate or fused to another ring, optionally containing at least one hetero atom, the alkyl radicals and the rings also possibly being substituted with at least one substituent A₁, with R₉ and R′₉ independently denoting H, a C₁-C₂₀ alkyl radical or a ring which is isolated or fused to another ring;
the rings containing 5 or 6 atoms;
the hetero atoms being O, N or S or a combination thereof.

12. Use according to one of the preceding claims, characterized in that at least one from among R₁ and R₂ represents a hydrogen atom, a halogen atom, OR₇ or CF₃.

13. Use according to one of the preceding claims, characterized in that R₁ and R₂ are located on the phenyl ring, in an ortho position to the branching of the pyrazole portion.

14. Use according to one of the preceding claims, characterized in that R₁ and/or R₂ represent(s) a halogen atom, especially a chlorine atom.

15. Use according to one of the preceding claims, characterized in that R₃ represents CN.

16. Use according to the preceding claim, characterized in that R₄, R₅ and R₆ represent, independently of each other, NH₂, H, CN, a C₁-C₁₀ alkyl radical optionally substituted with OR₁₀, or a saturated or unsaturated hydrocarbon-based ring containing 5 or 6 atoms.

17. Use according to one of the preceding claims, characterized in that R₆ represents CH₂CH₂OH or a phenyl radical.

18. Use according to one of the preceding claims, characterized in that R₄ represents NH₂ or H.

19. Use according to one of the preceding claims, characterized in that R₅ represents CN or H.

20. Use according to one of the preceding claims, characterized in that the styrylpyrazole compound is of formula (III) below, or a salt thereof: 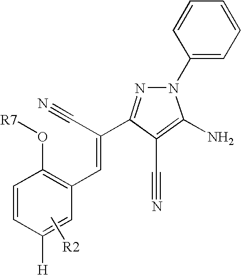
R₇ represents
a) a linear or branched, saturated or unsaturated C₁-C₁₀ alkyl radical, optionally substituted with at least one substituent A₁; or
b) a saturated or unsaturated ring C¹ of 4 to 7 atoms, optionally containing at least one hetero atom and/or being optionally substituted with at least one substituent A₁ and/or optionally fused to at least one saturated or unsaturated ring C² of 4 to 7 atoms, optionally containing at least one hetero atom;
R₂ represents
OR₇, SR₇, NR₇R′₇, COOR₇, CONR₇R′₇, CF₃, CN, NR₇COR′₇, SO₂R₇, SO₂NR₇R′₇, NR₇SO₂R′₇, COR₇, CSR₇, OCOR₇, COSR₇, SCOR₇, CSNR₇R′₇, NR₇CONR′₇R″₇, NR₇C(═NR′₇)NR″₇R″′₇, NR₇CSR′₇ and NR₇CSNR′₇R″₇, a saturated or unsaturated C₁-C₁₀ alkyl radical, a saturated or unsaturated ring C³, which is separate or fused to another ring C⁴, optionally containing at least one hetero atom, the alkyl radicals and the rings also possibly being substituted with at least one substituent A₁ in which R₇ and R′₇, which may be identical or different, denote:
a hydrogen atom or a linear or branched, saturated or unsaturated C₁-C₁₀ alkyl radical,
a C² aromatic ring optionally including at least one hetero atom, optionally substituted with at least one substituent A₂;
in which the hetero atoms are chosen from N, O and S and a combination thereof.

21. Use according to one of the preceding claims, characterized in that the salt of the compound of formula (I) is a salt chosen from the sodium and potassium salts, the zinc (Zn²⁺), calcium (Ca²⁺), copper (Cu²⁺), iron (Fe²⁺), strontium (Sr²⁺), magnesium (Mg²⁺), ammonium and manganese (Mn₂₊) salts, the triethanolamine, monoethanolamine, diethanolamine, hexadecylamine, N,N,N′,N′-tetrakis(2-hydroxypropyl)ethylenediamine and tris(hydroxymethyl)aminomethane salts, and the hydroxides, carbonates, sulphates, phosphates, halides and nitrates.

22. Use according to one of the preceding claims, characterized in that the compound of formula (I) is chosen from:

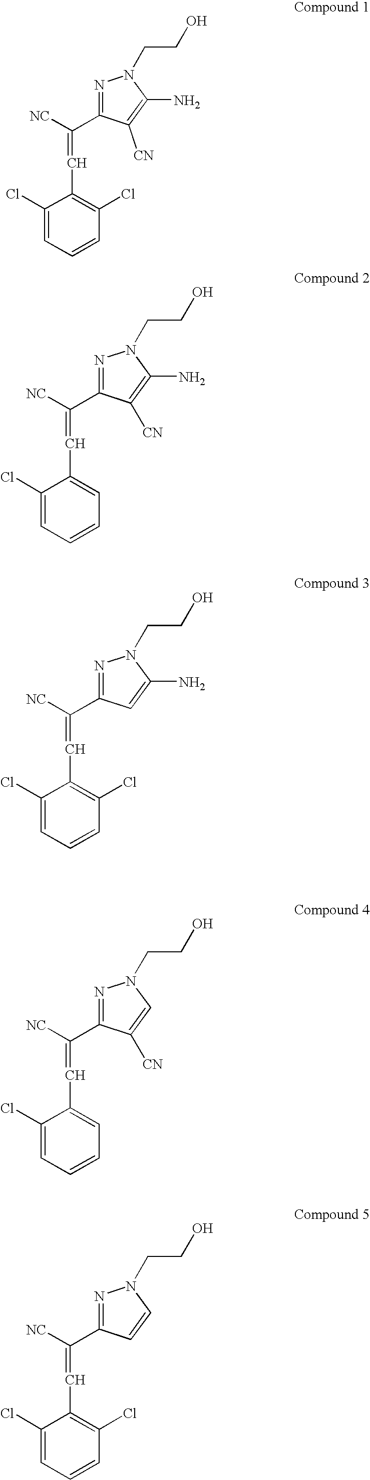

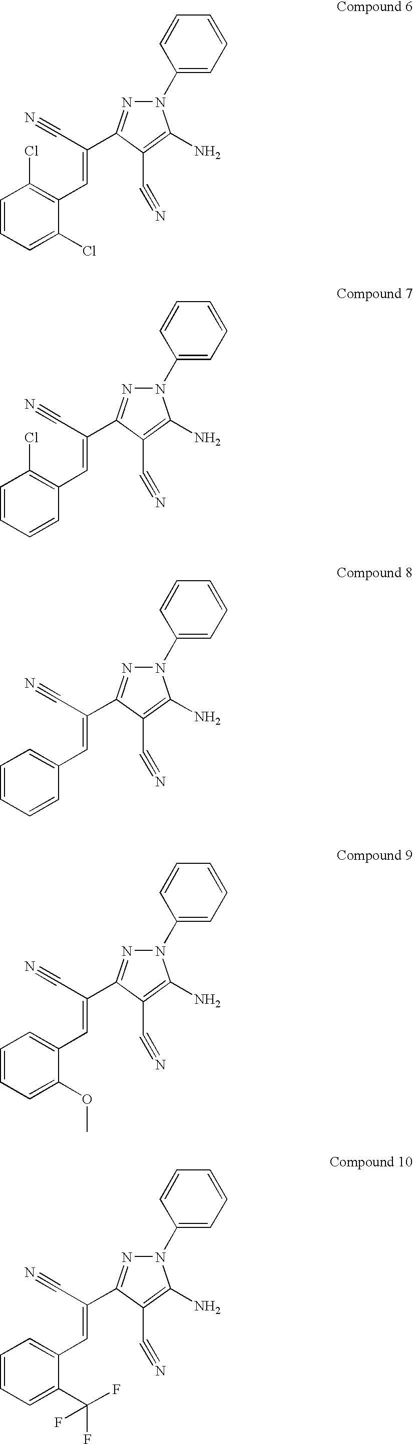

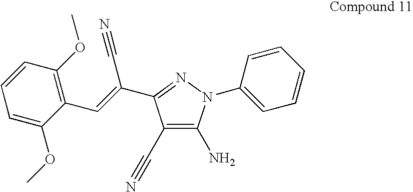

23. Use according to one of the preceding claims, characterized in that the compound of formula (I) or a mixture of compounds of formula (I) is used at a concentration ranging from 10⁻³% to 10% and preferably from 10⁻²% to 2% relative to the total weight of the composition.

24. Use according to one of claims 2, 3 and 5 to 23, characterized in that the composition is a composition for topical application.

25. Haircare or makeup composition for keratin fibres, containing a physiologically acceptable medium and an effective amount of at least one styrylpyrazole compound of formula (I), or a salt thereof: 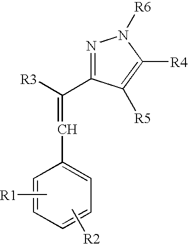
in which:
R₁, R₂, R₄ and R₅, which may be identical or different, are chosen from hydrogen, a halogen, groups OR₇, SR₇, NR₇R′₇, COOR₇, CONR₇R′₇, CF₃, CN, NR₇COR′₇, SO₂R₇, SO₂NR₇R′₇, NR₇SO₂R′₇, COR₇, CSR₇, OCOR₇, COSR₇, SCOR₇, CSNR₇R′₇, NR₇CONR′₇R″₇, NR₇C(═NR′₇)NR″₇R″′₇, NR₇CSR′₇ and NR₇CSNR′₇R″₇, saturated or unsaturated, linear or branched C₁-C₂₀ alkyl radicals, saturated or unsaturated rings of 4 to 7 atoms, optionally containing at least one hetero atom, these rings possibly being separate or fused, the alkyl radicals and the rings also possibly being substituted with at least one substituent A₁, with R₇, R′₇, R″₇ and R″′₇ independently denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₂;
R₃ is chosen from CN, COOR₈, CONR₈R′₈, COR₈, SO₂R₈ and SO₂NR₈R′₈, with R₈ and R′₈ independently denoting hydrogen, a linear or branched C₁-C₂₀ alkyl radical or a ring of 4 to 7 atoms, isolated or fused to another ring and optionally containing at least one hetero atom, the alkyl radical or the said rings being saturated or unsaturated and optionally substituted with at least one substituent A₃;
R₆ is chosen from hydrogen, groups COOR₉, COR₉, CSR₉, COSR₉, CONR₉R′₉, SO₂R₉ and SO₂NR₉R′₉, linear or branched, saturated or unsaturated C₁-C₂₀ alkyl radicals and saturated or unsaturated rings of 4 to 7 atoms, optionally containing at least one hetero atom, these rings possibly being separate or fused, the alkyl radicals and the rings also possibly being substituted with at least one substituent A₄, with R₉ and R′₉, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated and optionally substituted with at least one substituent A₅;
A₁, A₂, A₃, A₄ and A₅ being chosen independently from halogens, groups OR₁₀, SR₁₀, NR₁₀R′₁₀, COOR₁₀, CH₂COOR₁₀, CONR₁₀R′₁₀, CF₃, CN, NR₁₀COR′₁₀, SO₂R₁₀, SO₂NR₁₀R′₁₀, NR₁₀SO₂R′₁₀, COR₁₀, CSR₁₀, OCOR₁₀, COSR₁₀, SCOR₁₀, CSNR₁₀R′₁₀, NR₁₀CONR′₁₀R″₁₀, NR₁₀C(═NR′₁₀)NR″₁₀R″′₁₀, NR₁₀CSNR′₁₀R″₁₀ and NR₁₀CSR′₁₀, with R₁₀, R′₁₀, R″₁₀ and R″′₁₀, which may be identical or different, denoting hydrogen, a linear or branched C₁-C₂₀ alkyl radical or a ring of 4 to 7 atoms, optionally containing at least one hetero atom, isolated or fused to another ring, the alkyl radical or the said rings being saturated or unsaturated.

26. Composition according to claim 25, characterized in that the styrylpyrazole compound is of formula (II) below, or a salt thereof: 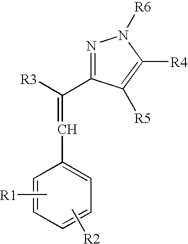
in which:
R₁, R₂, R₄ and R₅ independently represent H, a halogen, OR₇, SR₇, NR₇R′₇, COOR₇, CONR₇R′₇, CF₃, CN, a saturated or unsaturated C₁-C₁₀ alkyl radical, a saturated or unsaturated ring, separate or fused to another ring, optionally containing at least one hetero atom, the alkyl radicals and the rings also possibly being substituted with at least one substituent A₁, with R₇ and R′₇ independently denoting H, a C₁-C₁₀ alkyl radical or a ring which is isolated or fused to another ring;
R₃ represents CN, COOR₈, CONR₈R′₈ or COR₈, with R₈ and R′₈ independently denoting H, a C₁-C₁₀ alkyl radical or a ring which is isolated or fused to another ring and optionally containing at least one hetero atom, the said rings being saturated or unsaturated and optionally substituted with at least one substituent A₁;
R₆ represents hydrogen, COOR₉, COR₉, a saturated or unsaturated C₁-C₁₀ alkyl radical or a saturated or unsaturated ring, which is separate or fused to another ring, optionally containing at least one hetero atom, the alkyl radicals and the rings also possibly being substituted with at least one substituent A₁, with R₉ and R′₉ independently denoting H, a C₁-C₂₀ alkyl radical or a ring which is isolated or fused to another ring;
the rings containing 5 or 6 atoms;
the hetero atoms being O, N or S or a combination thereof.

27. Composition according to claim 25 or 26, characterized in that at least one from among R₁ and R₂ represents a hydrogen atom, a halogen atom, OR₇ or CF₃.

28. Composition according to one of claims 25 to 27, characterized in that R₁ and R₂ are located on the phenyl ring, in an ortho position to the branching of the pyrazole portion.

29. Composition according to one of claims 25 to 28, characterized in that R₁ and/or R₂ represent(s) a halogen atom, especially a chlorine atom.

30. Composition according to one of claims 25 to 29, characterized in that R₃ represents CN.

31. Composition according to one of claims 25 to 30, characterized in that R₄, R₅ and R₆ represent, independently of each other, NH₂, H, CN, a C₁-C₁₀ alkyl radical optionally substituted with OR₁₀, or a saturated or unsaturated hydrocarbon-based ring containing 5 or 6 atoms.

32. Composition according to one of claims 25 to 31, characterized in that R₆ represents CH₂CH₂OH or a phenyl radical.

33. Composition according to one of claims 25 to 32, characterized in that R₄ represents NH₂ or H.

34. Composition according to one of claims 25 to 33, characterized in that R₅ represents CN or H.

35. Composition according to one of claims 25 to 34, characterized in that the styrylpyrazole compound is of formula (III) below, or a salt thereof: 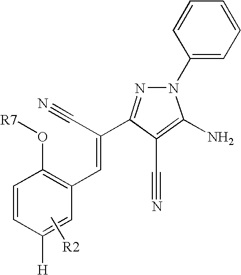
R₇ represents
a) a linear or branched, saturated or unsaturated C₁-C₁₀ alkyl radical, optionally substituted with at least one substituent A₁; or
b) a saturated or unsaturated ring C¹ of 4 to 7 atoms, optionally containing at least one hetero atom and/or being optionally substituted with at least one substituent A₁ and/or optionally fused to at least one saturated or unsaturated ring C of 4 to 7 atoms, optionally containing at least one hetero atom;
R₂ represents
OR₇, SR₇, NR₇R′₇, COOR₇, CONR₇R′₇, CF₃, CN, NR₇COR′₇, SO₂R₇, SO₂NR₇R′₇, NR₇SO₂R′₇, COR₇, CSR₇, OCOR₇, COSR₇, SCOR₇, CSNR₇R′₇, NR₇CONR′₇R″₇, NR₇C(═NR′₇)NR″₇R″′₇, NR₇CSR′₇ and NR₇CSNR′₇R″₇, a saturated or unsaturated C₁-C₁₀ alkyl radical, a saturated or unsaturated ring C³, which is separate or fused to another ring C⁴, optionally containing at least one hetero atom, the alkyl radicals and the rings also possibly being substituted with at least one substituent A₁ in which R₇ and R′₇, which may be identical or different, denote:
a hydrogen atom or a linear or branched, saturated or unsaturated C₁-C₁₀ alkyl radical,
a C² aromatic ring optionally including at least one hetero atom, optionally substituted with at least one substituent A₂; and
in which the hetero atoms are chosen from N, O and S and a combination thereof.

36. Composition according to one of claims 25 to 35, characterized in that the salt of the compound of formula (I) is a salt chosen from the sodium and potassium salts, the zinc (Zn²⁺) , calcium (Ca²⁺), copper (Cu²⁺), iron (Fe²⁺), strontium (Sr²⁺), magnesium (Mg²⁺) , ammonium and manganese (Mn²⁺) salts, the triethanolamine, monoethanolamine, diethanolamine, hexadecylamine, N,N,N′,N′-tetrakis(2-hydroxypropyl)ethylenediamine and tris(hydroxymethyl)aminomethane salts, and the hydroxides, carbonates, sulphates, phosphates, halides and nitrates.

37. Composition according to one of claims 25 to 36, characterized in that the compound of formula (I) is chosen from:

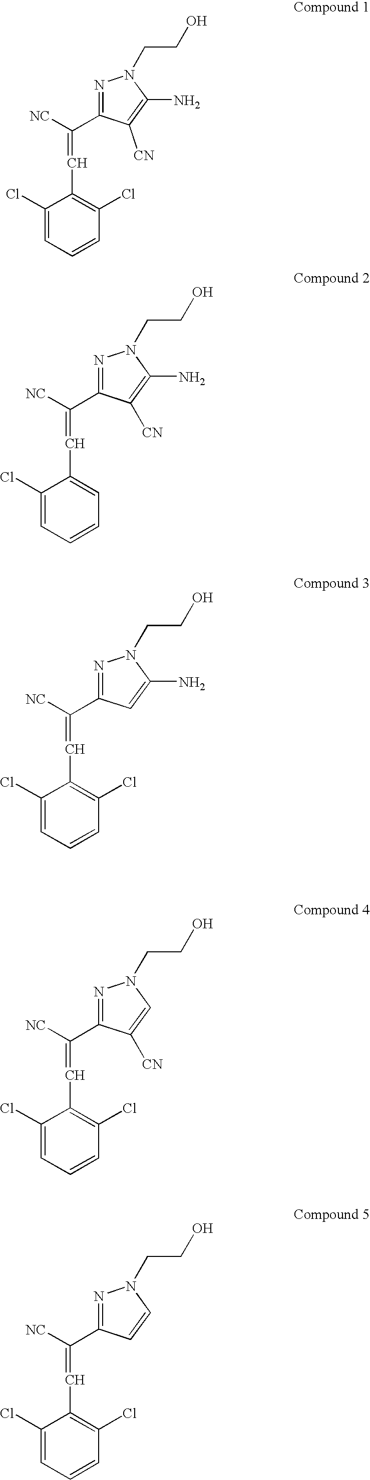

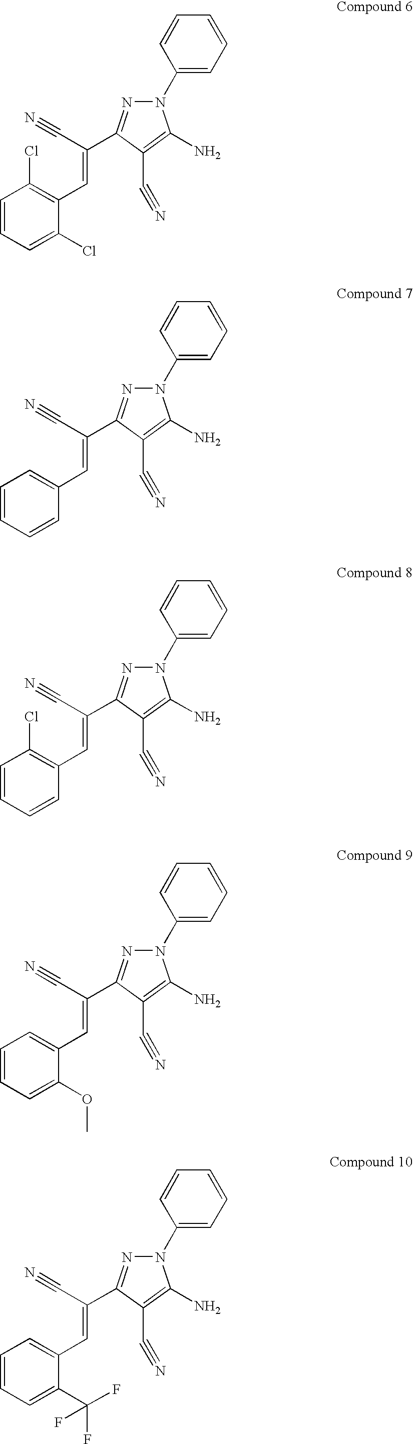

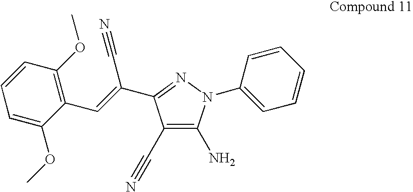

38. Composition according to one of claims 25 to 37, characterized in that the compound of formula (I) or a mixture of compounds of formula (I) is used at a concentration ranging from 10⁻³% to 10% and preferably from 10⁻²% to 2% relative to the total weight of the composition.

39. Composition according to one of claims 25 to 38, characterized in that it is in the form of a hair cream, a hair lotion, a shampoo, a conditioner or a mascara for the hair or the eyelashes.

40. Composition according to one of claims 25 to 39, characterized in that it is in the form of an aqueous, alcoholic or aqueous-alcoholic solution or suspension.

41. Composition according to one of claims 25 to 40, characterized in that it contains other ingredients chosen from solvents, aqueous-phase or oily-phase thickeners or gelling agents, dyestuffs that are soluble in the medium of the composition, fillers, pigments, antioxidants, preserving agents, fragrances, electrolytes, neutralizers, film-forming polymers, UV-blockers and cosmetic and pharmaceutical active agents, and mixtures thereof.

42. Composition according to one of claims 25 to 41, characterized in that it also contains another active agent chosen from proteins, protein hydrolysates, amino acids, polyols, urea, allantoin, sugars and sugar derivatives, plant extracts, hydroxy acids, retinol derivatives, tocopherol derivatives, essential fatty acids, ceramides, essential oils, salicylic acid and its derivatives, for instance 5-n-octanoyl salicylic acid, hydroxy acid esters and phospholipids.

43. Composition according to one of claims 25 to 42, characterized in that it contains at least one additional active compound that promotes the regrowth and/or limits the loss of keratin fibres.

44. Composition according to one of claims 25 to 43, characterized in that it contains at least one additional active compound that promotes the regrowth and/or limits the loss of keratin fibres, chosen from aminexil, 6-O-[(9Z,12Z)octadeca-9,12-dienoyl]hexapyranose, lipoxygenase inhibitors, bradykinin inhibitors, prostaglandins and derivatives thereof, prostaglandin receptor agonists or antagonists, non-prostanoic prostaglandin analogues, vasodilators, antiandrogens, cyclosporins and analogues thereof, antimicrobial agents, anti-inflammatory agents, retinoids, benzalkonium chloride, benzethonium chloride, phenol, oestradiol, chlorpheniramine maleate, chlorophylline derivatives, cholesterol, cysteine, methionine, menthol, peppermint oil, calcium pantothenate, panthenol, resorcinol, protein kinase C activators, glycosidase inhibitors, glycosaminoglycanase inhibitors, pyroglutamic acid esters, hexosaccharidic or acylhexosaccharidic acids, aryl-substituted ethylenes, N-acyl amino acids, flavonoids, ascomycin derivatives and analogues, histamine antagonists, saponins, proteoglycanase inhibitors, oestrogen agonists and antagonists, pseudoterines, cytokines and growth factor promoters, IL-1 or IL-6 inhibitors, IL-10 promoters, TNF inhibitors, benzophenones, hydantoin, octopirox, retinoic acid, antipruriginous agents, antiparasitic agents, antifungal agents, nicotinic acid esters, calcium antagonists, hormones, triterpenes, antiandrogens, steroidal or non-steroidal 5-α-reductase inhibitors, potassium-channel agonists and FP receptor agonists, and mixtures thereof.

45. Composition according to claim 44, characterized in that the additional compound is chosen from aminexil, FP receptor agonists and vasodilators.

46. Care or makeup composition for keratin fibres, comprising, in a physiologically acceptable medium, in particular a cosmetic medium, at least one compound of formula (I), or a salt thereof, and at least one additional active compound for promoting the regrowth of human keratin fibres and/or for limiting their loss, chosen from aminexil, FP receptor agonists and vasodilators.

47. Composition according to one of claims 43 to 46, characterized in that the additional active compound is chosen from aminexil, minoxidil, latanoprost, butaprost and travoprost.

48. Cosmetic process for treating keratin fibres and/or the skin from which the said fibres emerge, characterized in that it consists in applying to the fibres and/or the skin a cosmetic composition as defined in any of claims 25 to 47, leaving this composition in contact with the fibres and/or the skin, and optionally rinsing it out.

49. Cosmetic care and/or makeup process for human eyelashes, to improve their condition and/or appearance, characterized in that it consists in applying to the eyelashes and/or the eyelids a mascara composition comprising at least one compound of formula (I) or a salt thereof, and leaving this composition in contact with the eyelashes and/or the eyelids.

50. Cosmetic care process for human hair and/or the scalp, to improve their condition and/or appearance, characterized in that it consists in applying to the hair and/or the scalp a cosmetic composition as defined in any one of claims 25 to 47, leaving the composition in contact with the hair and/or the scalp, and optionally rinsing it out.

51. Styrylpyrazole compound of formula (III) below, or a salt thereof: 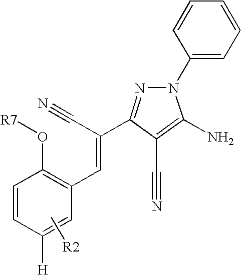
R₇ represents
a) a linear or branched, saturated or unsaturated C₁-C₁₀ alkyl radical, optionally substituted with at least one substituent A₁; or
b) a saturated or unsaturated ring C¹ of 4 to 7 atoms, optionally containing at least one hetero atom and/or being optionally substituted with at least one substituent A₁ and/or optionally fused to at least one saturated or unsaturated ring C² of 4 to 7 atoms, optionally containing at least one hetero atom;
R₂ represents
OR₇, SR₇, NR₇R′₇, COOR₇, CONR₇R′₇, CF₃, CN, NR₇COR′₇, SO₂R₇, SO₂NR₇R′₇, NR₇SO₂R′₇, COR₇, CSR₇, OCOR₇, COSR₇, SCOR₇, CSNR₇R′₇, NR₇CONR′₇R″₇, NR₇C(═NR′₇)NR″₇R″′₇, NR₇CSR′₇ and NR₇CSNR′₇R″₇, a saturated or unsaturated C₁-C₁₀ alkyl radical, a saturated or unsaturated ring C³, which is separate or fused to another ring C⁴, optionally containing at least one hetero atom, the alkyl radicals and the rings also possibly being substituted with at least one substituent A₁ in which R₇ and R′₇, which may be identical or different, denote:
a hydrogen atom or a linear or branched, saturated or unsaturated C₁-C₁₀ alkyl radical,
a C² aromatic ring optionally including at least one hetero atom, optionally substituted with at least one substituent A₂; and
in which the hetero atoms are chosen from N, O and S and a combination thereof.

52. Compound according to claim 51, characterized in that R₂ represents OR₇ and R₇ represents a saturated C₁-C₁₀ alkyl radical.
